(19) United States
(12) Patent Application Publication
(10) Pub. No.: US 2024/0424005 A1
(43) Pub. Date: Dec. 26, 2024

(54) DIAGNOSING, MONITORING AND TREATING NEUROLOGICAL DISEASE WITH PSYCHOACTIVE TRYPTAMINE DERIVATIVES AND mRNA MEASUREMENTS

(71) Applicant: NOVA MENTIS LIFE SCIENCE CORP., Vancouver (CA)

(72) Inventors: Marvin S. Hausman, Chuluota, FL (US); Julia V. Perederiy, Porter Ranch, CA (US); Kyle H. Ambert, Portland, OR (US); Viviana Trezza, Rome (IT)

(21) Appl. No.: 18/705,375

(22) PCT Filed: Nov. 8, 2022
(86) PCT No.: PCT/CA2022/051650

§ 371 Date: Apr. 26, 2024

Related U.S. Application Data
(60) Provisional application No. 63/341,380, filed on May 12, 2022.
(60) Provisional application No. 63/276,976, filed on Nov. 8, 2021.

(51) Int. Cl. A61K 31/675; A61P 25/00; A61P 25/28; C12Q 1/6883

(52) U.S. Cl. CPC A61K 31/675; A61P 25/00; A61P 25/28; C12Q 1/6883; C12Q 2600/106; C12Q 2600/158

(57) ABSTRACT

Provided is a method of treating a disease or disorder in a patient. The method comprises administering a psychoactive tryptamine derivative to the patient in a manner sufficient to treat the disease or disorder. In these embodiments, the disease or disorder is a neurodevelopmental disease, a neurodegenerative disease, a neurometabolic disease or anxiety.

Also provided is a method of diagnosing or monitoring progression or treatment of a disease or disorder in a patient, where the disease or disorder is a neurodevelopmental disease, a neurodegenerative disease, a neurometabolic disease or anxiety.

Additionally provided is a method of developing a treatment of a disease or disorder in a patient, where the disease or disorder is a neurodevelopmental disease, a neurodegenerative disease, a neurometabolic disease or anxiety. The method comprises
treating more than one patient having the disease or disorder with the treatment, and
determining mRNA levels in the patient of one or more genes for an inflammatory cytokine, a cytokine target, neurotransmission, serotonin signaling, a membrane channel, DNA damage repair, a growth factor, A2AP, CAPG, and/or NHBS, wherein
if the mRNA levels indicate that the treatment alleviates a symptom of the disease or disorder, development of the treatment is continued, and
if the mRNA levels indicate that the treatment does not alleviate a symptom of the disease or disorder, development of the treatment is discontinued.

### CROSS-REFERENCE TO RELATED APPLICATION

This application claims the benefit of U.S. Provisional Application No. 63/276,976, filed Nov. 8, 2021, and U.S. Provisional Application No. 63/341,380, filed May 12, 2022, both incorporated by reference herein in their entirety.

### BACKGROUND OF THE INVENTION

### (1) Field of the Invention

The present application generally relates to diagnosis, monitoring and treating neuroinflammatory diseases and conditions. More specifically, treatment of neuroinflammatory diseases and conditions with psychoactive tryptamine derivatives is provided, as is methods of diagnosis, monitoring, treating neuroinflammatory diseases and conditions, and methods of developing a treatment of neuroinflammatory diseases and conditions.

### (2) Description of the Related Art

Neuro- and systemic inflammation are known causes or comorbidities of various metabolic and neurological conditions, but this knowledge has not yet been applied in the clinic.

### BRIEF SUMMARY OF THE INVENTION

Provided is a method of treating a disease or disorder in a patient. The method comprises administering a psychoactive tryptamine derivative to the patient in a manner sufficient to treat the disease or disorder. In these embodiments, the disease or disorder is a neurodevelopmental disease, a neurodegenerative disease, a neurometabolic disease or anxiety.

Also provided is a method of diagnosing or monitoring progression or treatment of a disease or disorder in a patient, where the disease or disorder is a neurodevelopmental disease, a neurodegenerative disease, a neurometabolic disease or anxiety. The method comprises

determining mRNA levels in the patient of one or more genes for an inflammatory cytokine, a cytokine target, neurotransmission, serotonin signaling, a membrane channel, DNA damage repair, a growth factor, A2AP, CAPG, and/or NHBS, and

treating the patient for the disease or disorder if the mRNA levels indicate that the patient has the disease or disorder, or

continuing treating the patient for the disease or disorder if the mRNA levels indicate that the patient is responding to the treatment of the disease or disorder.

Additionally provided is a method of developing a treatment of a disease or disorder in a patient, where the disease or disorder is a neurodevelopmental disease, a neurodegenerative disease, a neurometabolic disease or anxiety. The method comprises

treating more than one patient having the disease or disorder with the treatment, and

determining mRNA levels in the patient of one or more genes for an inflammatory cytokine, a cytokine target, neurotransmission, serotonin signaling, a membrane channel, DNA damage repair, a growth factor, A2AP, CAPG, and/or NHBS, wherein

if the mRNA levels indicate that the treatment alleviates a symptom of the disease or disorder, development of the treatment is continued, and

if the mRNA levels indicate that the treatment does not alleviate a symptom of the disease or disorder, development of the treatment is discontinued.

### BRIEF DESCRIPTION OF THE SEVERAL VIEWS OF THE DRAWINGS

FIG. 1 is an illustration of the effects of systemic inflammation on microglia. From Perry and Holmes, 2014.

FIG. 2 is an illustration showing initiators of obesity-associated inflammation in adipocytes. From Saad et al., 2016.

FIG. 3 is an illustration of the architecture and neuroimmune cell types in white (WAT) and brown adipose tissue (BAT) in the basal state. From Blaszkiewicz et al., 2019.

FIG. 4 is an illustration showing that mitochondrial dysfunction affects diverse cellular processes that can culminate in cell death. From Henchcliffe and Beal, 2008.

FIG. 5 is an illustration showing plasticity in vagal afferent neurons. From de Lartigue, 2016.

FIG. 6 is an illustration showing a pathogen-activated immune response and the subsequent production of signals. From Srinivasan et al., 2016.

FIG. 7 is an illustration showing an overview over the basic anatomy and functions of the vagus nerve. From Breit et al., 2018.

FIG. 8 shows timelines of the experiments described in Example 3.

FIG. 9 is graphs showing results of Experiment 1 of Example 3, demonstrating that acute intraperitoneal administration of psilocybin mitigates the cognitive deficits displayed by Fmr1-^{Δ}exon 8 rats. A single administration of psilocybin, at the dose of 1 or 3 mg/kg, mitigated the cognitive deficit (expressed as lower discrimination index in Panel A) displayed by Fmr1-^{Δ}exon 8 rats in the object recognition task (WT-VEH n=11, WT-PSY 1 mg/kg n=10, WT-PSY 0.3 mg/kg n=14, KO-VEH n=13, KO-PSY 1 mg/kg n=13; KO-PSY 3 mg/kg n=14). In the experiment, no significant differences were found in the total time spent by animals exploring the objects (Panel B), indicating an intact exploratory activity in all experimental groups.

FIG. 10 is graphs showing results of Experiment 2 of Example 3, demonstrating that repeated intraperitoneal administration of psilocybin normalizes the cognitive deficits displayed by Fmr1-^{Δ}exon 8 rats. Repeated microdoses (0.1 mg/kg) of psilocybin reverted the deficit in object recognition memory displayed by Fmr1-^{Δ}exon 8 rats (WT-VEH n=10, WT-PSY n=9, KO-VEH n=9, KO-PSY n=8). In the experiment, no significant differences were found in the total time spent by animals exploring the objects (Panel B), indicating an intact exploratory activity in all experimental groups.

FIG. 11 is graphs showing results of Experiment 3 of Example 3, demonstrating that repeated oral administration of psilocybin reverts the cognitive deficits displayed by Fmr1-^{Δ}exon 8 rats. Repeated oral administration of microdoses (0.1 or 0.3 mg/kg) of psilocybin fully reverted the deficit in object recognition memory displayed by Fmr1-^{Δ}exon 8 rats (WT-VEH n=9, WT-PSY 0.1 n=9, WT PSY 0.3=9, KO-VEH n=8, KO-PSY 0.1 n=10, KO-PSY 0.3=10). In the experiment, no significant differences were found in the total time spent by animals exploring the objects (Panel B), indicating an intact exploratory activity in all experimental groups.

FIG. 12 shows the timeline utilized in the experiments in Example 4.

FIG. 13 is graphs showing the effect of psilocybin (1 mg/kg, 1 day before testing) in the elevated plus maze test.

### DETAILED DESCRIPTION OF THE INVENTION

The present invention provides new tools to help with accurate diagnosis and disease monitoring, especially in complex neuroinflammatory diseases and disorders without available effective treatments, such as various neurodevelopmental, neurodegenerative, and neurometabolic diseases.

Biomarkers are indicators of specific biological activity and thereby provide an opportunity to establish a characteristic biochemical profile of disease pathogenesis, progression, and/or treatment. Three sources for novel genetic biomarkers of neuroinflammatory conditions are utilized: (I) immune cells & cytokines, (II) metabolic signals, and (III) serotonergic signaling. Biomarker panels are customized to each of the following groups of neuroinflammatory conditions: (A) neurodevelopmental diseases such as autism spectrum disorders, attention deficit hyperactivity disorder (ADHD) and fragile X syndrome, (B) neurodegenerative diseases such as Alzheimer's disease, Parkinson's disease, multiple sclerosis, and amyotrophic lateral sclerosis, and (C) neurometabolic diseases such has obesity, type 1 diabetes and type 2 diabetes. Although diverse, these conditions are all associated with increased activation of immune cells, increased levels of inflammatory cytokines/chemokines, and altered neural/cellular/hormonal signaling, particularly along the brain-gut axis.

In one aspect, the present invention provides a method of treating several diseases and conditions having a neuroinflammatory etiology with a psychoactive tryptamine derivative.

Thus, in some embodiments, a method of treating a disease or disorder in a patient is provided. The method comprises administering a psychoactive tryptamine derivative to the patient in a manner sufficient to treat the disease or disorder. In these embodiments, the disease or disorder is a neurodevelopmental disease, a neurodegenerative disease, a neurometabolic disease or anxiety.

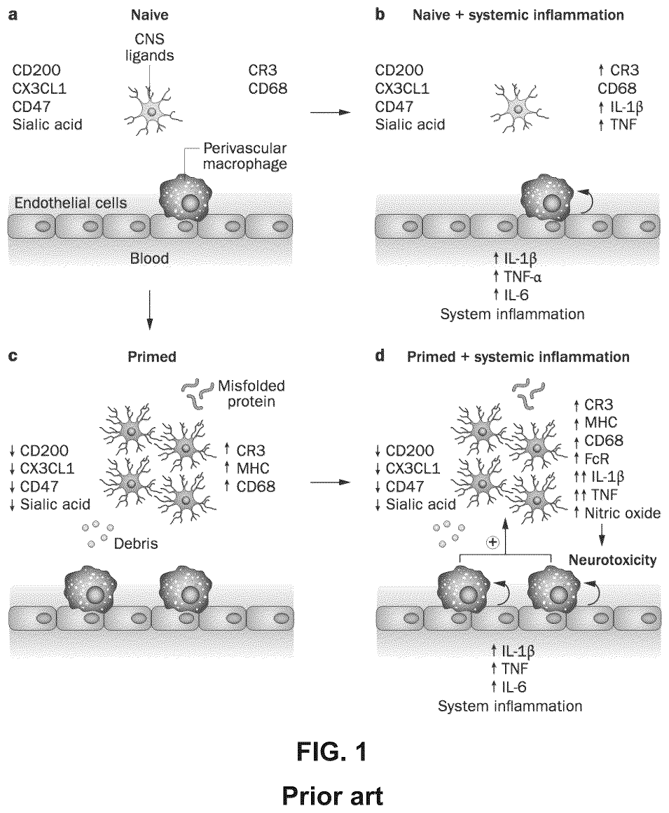

These methods can utilize any psychoactive tryptamine derivative now known or later discovered. As used herein, a psychoactive tryptamine derivative is a psychoactive compound having a tryptamine backbone, including naturally occurring compounds, e.g., produced by Psilocybe spp., such as norbaeocystin, baeocystin, psilocybin or psilocin (see, e.g., FIG. 1 of WO 2019/180309), 5-methoxy-N,N-dimethyltryptamine, dimethyltryptamine (a major active ingredient in ayahuasca, a psychoactive brew made from the Banisteriopsis caapi vine or the Psychotria viridis shrub), 5-hydroxy-N,N-dimethyltryptamine (bufotenine, produced in skin secretions of Brazilian Rhinella toads), or synthetic compounds, such as the dozens of compounds described in Malaca et al. (2020), including α-methyltryptamine, α-ethyltryptamine, diethyltryptamine, N-methyl-N-ethyltryptamine, dipropyltryptamine, diisopropyltryptamine, 4-hydroxy-N-methyl-N-ethyltryptamine, 4-hydroxy-N,N-dipropyltryptamine, 4-acetoxy-N,N-dipropyltryptamine, 4-acetyloxy-N,N-diallyltryptamine, 4-acetoxy-N-methyl-N-ethyltryptamine (matacetin), 5-methoxy-α-methyltryptamine, 5-methoxy-N, N-dipropyltryptamine, 5-methoxy-N, N-diisopropyltryptamine, 5-methoxy-N,N-diallyltryptamine, 5-methoxy-N-methyl-N-isopropyltryptamine, 5-methoxy-N-methyl-N-ethyltryptamine, and 5-methoxy-N,N-trimethyltryptamine. In some embodiments, the psychoactive tryptamine derivative is psilocybin baeocystin, aeruginascin, or psilocin.

The psychoactive tryptamine derivative can be administered in any dose. See, e,g, Nicolas et al., 2018; Kuypers, 2020; Garcia-Romeu et al., 2021; Griffiths et al., 2011.

In some embodiments, the psychoactive tryptamine derivative is administered at a dose where psychoactive effects are perceived by the patient, e.g., 3, 5, 10, 15, 20, 25, 30, 40, 50, 60 mg, or more, or any amount in between those amounts, depending on the psychedelic tryptamine derivative administered. In other embodiments, the psychoactive tryptamine derivative is administered in a dose below which psychoactive effects are perceived by the patient (“microdosing”), e.g., less than 3, 2, 1, 0.5, 0.2, 0.1 mg, or any amount in between. The dosage can be administered based on the weight of the patient, or as a set dose regardless or weight (see Garcia-Romeu et al., 2021).

The psychoactive tryptamine derivative can be administered by any route, e.g., parenteral (intramuscular, intraperitoneal, intravenous (IV) or subcutaneous injection), enteral (oral), transmucosal (nasal, vaginal, rectal, or sublingual), or transdermal (e.g., through a patch). Methods for formulating the psychoactive tryptamine derivative to be administered by any of those routes are known in the art.

In some of these embodiments, the disease or disorder is a neurodevelopmental disease, for example autism spectrum disorder, ADHD, or fragile X syndrome. In further embodiments, the disease or disorder is a neurodegenerative disease, including but not limited to Alzheimer's disease, Parkinson's disease, multiple sclerosis, or amyotrophic lateral sclerosis. In other embodiments, the disease or disorder is a neurometabolic disease, e.g., obesity, type 1 diabetes or type 2 diabetes. In additional embodiments, the disease or disorder is anxiety. See Example 4, demonstrating that psilocybin alleviates anxiety.

Also provided is a method of diagnosing or monitoring progression or treatment of a disease or disorder in a patient, where the disease or disorder is a neurodevelopmental disease, a neurodegenerative disease, a neurometabolic disease or anxiety. The method comprises

determining mRNA levels in the patient of one or more genes for an inflammatory cytokine, a cytokine target, neurotransmission, serotonin signaling, a membrane channel, DNA damage repair, a growth factor, A2AP, CAPG, and/or NHBS, and

treating the patient for the disease or disorder if the mRNA levels indicate that the patient has the disease or disorder, or

continuing treating the patient for the disease or disorder if the mRNA levels indicate that the patient is responding to the treatment of the disease or disorder.

This method leverages differential expression of RNAs as a basis for a novel diagnostic and monitoring method for neuroinflammatory conditions, including neurodevelopmental diseases, neurodegenerative diseases, and neurometabolic diseases, are prevalent, with no effective solutions. Though these conditions have different etiologies, they often share causes and/or comorbidities. The mRNA panel reflects possible changes in cytokines and their targets, membrane proteins/channels and neurotransmission, serotonin signaling, DNA damage repair, and neurotrophic factors. Alternative embodiments include indicators of low-grade systemic inflammation, metabolic disruptions, altered serotonergic signaling, and exosomes. Table 1 shows the involvement of (I) immune cells and cytokines, (II) metabolic signals, and (III) serotonergic signaling in those diseases and disorders.

TABLE 1
mRNA Biomarkers of Neural and Systemic Inflammation

| | (I) Immune cells & | (II) Metabolic | (III) Serotonergic |
|---|---|---|---|
| | cytokines | signals | signaling |
| (A) | Microglia, autoantibodies, | mitochondrial dysfunction; | activity-dependent cellular |
| Neurodevelopmental | production of | relationship between | pathways that underlie |
| Diseases | neuroinflammatory | impaired energy and | circuit plasticity, 5-HT |
| | mediators: cytokines, | oxidative metabolic | receptors |
| | chemokines | pathways in the etiology | |
| | | of ASD | |
| (B) | Activated microglia, | Insulin resistance, | Serotonin 2A Receptor |
| Neurodegenerative | astrocytes, neurons, T- | GLP-1/GIP, impaired | Autoantibodies, |
| Diseases | cells, mast cells | brain metabolism, Type 2 | biomarkers of depression |
| | | diabetes markers | |
| (C) | Adipose tissue resident | markers of appetite, body | 5-HT receptors in |
| Neurometabolic | immune cells; polarization | weight, blood pressure, | metabolic homeostasis (5- |
| Diseases | of macrophages | glucose homeostasis, | HT_{1B}, 5-HT_{1F}, 5-HT_{2A-C}, 5- |
| | | insulin, liver injury, vagus | HT₃ and 5-HT₆) |
| | | nerve, ghrelin, leptin | |

Determination of the level of any one or combination of the following mRNAs provide insight into the diagnosis or monitoring of progression of the subject diseases or disorders: Cytokines—eotaxin, eotaxin-2, eotaxin-3, IL-5, MCP-1, MIP-1α, GROα, RANTES, MCP-3, IL-1β, IL-6, IL-8, IL-12A, TGF-β1, TNF-α, IL-4, IL-1β, IL-10
Cytokine targets—CCR3, NOD1, NLRA, IFNGR, HAAO
Neurotransmission—NMDAR1, GRIN2B, GRIN3A, GRIN2A, TLR-4, TLR3, TLR1, MAOA
Serotonin signaling—TPH1, TPH2, AADAT
Membrane channel—OCTN1, P2X4, P2X7, VDAC3, SLC22A15, SLC22A3, SLC3A2
DNA damage repair—ZNF365, TRIM26, ZNF827
Growth factor—GDNF, PDGF-A, NGF, FGF-13, GIF
   The Rationale for these mRNA Determinations is as Follows:

### (I) Immune Cells and Cytokines/Chemokines

The immune system is a complex network of defense mechanisms triggered to protect an organism from disease or pathogens like bacteria, viruses, fungi, and parasites. The immune response can be classified into the early-stage innate immunity and the later-stage adaptive immunity. Innate immunity is a first-line defense system within epithelial, mucosal, and glandular tissues, designed to produce a localized but non-specific effect to neutralize the invading pathogen via phagocytosis, production of antimicrobial substances, and activation of cytokine/chemokine production to help recruit cells, molecules, and fluid to the sites of infection—a process known as inflammation (Owen et al., 2013). Adaptive immunity is a more comprehensive and targeted response that occurs several days after the initial exposure to the pathogen or breach of physical barriers and involves an antibody response originating in the bone marrow (driven by B cells) and a cell-mediated response originating in the thymus (driven by T cells). B and T cells subsequently function as immune memory and facilitate prompt removal of known pathogens.

The immune system is in strictly regulated homeostasis of pro- and anti-inflammatory signaling, mediated by cytokines and chemokines, which are small molecules that facilitate local and systemic cellular communication via concentration gradients or endocrine function. Six cytokine families have been described, classified by molecular structure and type of receptors (Table 2).

TABLE 2
Details of six cytokine families and examples of family members (Masi et al., 2017)

| Cytokine Family | Examples | Characteristics |
|---|---|---|
| Interleukin 1 family | IL-1α, IL-β, IL-RA | Induce responses against infection that are primarily pro- |
| | | inflammatory |
| | | IL-RA antagonizes effects of IL-1α and IL-β |
| Hematopoietin (Class | IL-2, IL-4, IL-5, IL-6, IL-7, | Largest family of cytokines, all members sharing a 4-helix |
| I cytokine) family | IL-9, IL-12p40, IL-12p70, | bundle structure |
| | IL-13, IL-15, IL-23, | Sequence and functional diversity |
| | GM-CSF, G-CSF | Diverse effects include proliferation, differentiation, and |
| | | antibody secretion |
| Interferon (Class II | IFN-γ, IL-10 | Mediate early antiviral responses |
| cytokine) family | | Activate macrophages, interact with cells of the adaptive |
| | | immune system, and support the generation of Thi cells |
| Tumor necrosis factor | TNF-α, TNF-β1 | Expressed in either soluble or membrane-bound form |
| family | | Cause apoptosis |
| Interleukin 17 family | IL-17 | Primarily pro-inflammatory |
| Chemokine family | IL-8, Eotaxin, IP-10 (CXCL10), | Secondary pro-inflammatory mediators |
| | MCP-1(CCL2), MIP-1α (CCL3), | Stimulate recruitment of well-defined leukocyte subsets |
| | MIP-1β (CCL4), RANTES (CCL5) | Promote chemoattraction, movement of immune system cells |
| | | into, within, and out of lymphoid organs |

CCL, chemokine (C—C motif) ligand;
CXCL, chemokine (C-X-C) ligand;
G-CSF, granulocyte colony-stimulating factor;
GM-CSF, granulocyte macrophage colony-stimulating factor;
IFN-γ, interferon-γ;
IL, interleukin;
IL.1RA, IL-1 receptor antagonist;
IP-10, IFNγ-inducible protein 10;
MCP-1, monocyte chemotactic protein-1;
MIP-1α, macrophage inflammatory protein-1α;
MIP-1β, macrophage inflammatory protein-1β;
RANTES, regulated on activation, normal T cell expressed and secreted;
Th, T helper;
TNF-α, tumor necrosis factor-α.

Disruptions of the immune system present as chronic inflammation or immune deficiency, with symptoms ranging from allergies and asthma to gut disruptions and autoimmune conditions. In neuroinflammatory conditions such as autism, neurodegeneration, or obesity the immune system is chronically disrupted, and low-grade systemic inflammation is a common comorbidity.

### (I-A) Immune Cells and Cytokines/Chemokines: Neurodevelopmental Diseases

Immune dysfunction and chronic inflammation are a daily part of autism spectrum disorder (ASD) and may contribute to the expression of neurodevelopmental deficits of ASD and Fragile X Syndrome (FXS) (DiMarco et al., 2016). Autoimmune conditions are common in ASD and brain-specific autoantibodies have been detected in sera of autistic children, fueling an imbalance of cytokines and, often, an inappropriate immune response to environmental factors like infections or exposure to toxins. Cytokine and chemokine aberrations have been identified in ASD, with ties to the severity of cognitive outcomes (Ashwood et al., 2011). Though precise changes remain to be standardized, cytokine/chemokine aberrations in ASD include significantly higher concentrations of interleukin (IL)-1β, IL-6, IL-8, eotaxin, interferon-γ, IL-12p70, monocyte chemotactic protein-1, CCL2, and CCL5, with a decrease in transforming growth factor-β1 and CXCL9 (Heuer et al., 2019; Masi et al., 2015; Ashwood et al., 2008; Han et al., 2017). These biomarkers also correlate with deficits in cognitive function, likely by influencing essential neural plasticity mechanisms during brain development.

### (I-B) Immune Cells and Cytokines/Chemokines: Neurodegenerative Diseases

The immune response in the brain is largely mediated by microglia—the macrophage of the central nervous system. Microglia proliferate and adopt an activated state in response to inflammatory stimuli. Local triggers like neurodegeneration, accumulation of misfolded proteins ‘prime’ microglia—raising the baseline levels of chronic inflammation. This hypersensitive microglial state readily responds to a secondary stimulus such as systemic inflammation, mounting an exaggerated immune response and causing further neural damage (FIG. 1, from Perry and Holmes, 2014).

Activated microglia, as well as astrocytes, neurons, T-cells, and mast cells release inflammatory mediators of neuroinflammation and neurodegeneration. These include interleukin-1β (IL-1β), IL-6, IL-8, IL-33, tumor necrosis factor-α (TNF-α), chemokine (C—C motif) ligand 2 (CCL2), CCL5, matrix metalloproteinase (MMPs), granulocyte macrophage colony-stimulating factor (GM-CSF), glia maturation factor (GMF), substance P, reactive oxygen species (ROS), reactive nitrogen species (RNS), mast cells-mediated histamine and proteases, protease activated receptor-2 (PAR-2), CD40, CD40L, CD88, intracellular Ca+ elevation, and activation of mitogen-activated protein kinases (MAPKs) and nuclear factor kappa-B (NF-kB) (Kempuraj et al., 2016). Inflammatory and neurotoxic signaling originates from within the brain as well as from the periphery. Sustained systemic inflammation can affect the permeability of the blood-brain barrier (BBB)—an effect driven by activated microglia and their CCR5-dependent migration to the cerebral vasculature (Haruwaka et al., 2019). Defects in the BBB further expose sensitive neural tissue to pathogens from the peripheral environment.

FIG. 1 shows the effects of systemic inflammation on microglia (from Perry and Holmes, 2014). As illustrated in Panel a of FIG. 1, in the absence of disease, microglia use motile processes to survey the parenchyma, and are kept in the quiescent state by regulatory factors in the brain parenchymal microenvironment and inhibitory ligands expressed on the surface of neurons. As Panel b illustrates, following an initial systemic inflammatory stimulus, cytokines and other inflammatory mediators in the blood signal to receptors on cerebral endothelial cells, which in turn signal to perivascular macrophages and subsequently to microglia. These stimuli transiently activate microglia, leading to release of cytokines and probably other molecules in the brain that induce sickness behaviors. As illustrated in Panel c, during chronic neurodegeneration, neurotoxic misfolded proteins and debris from dysfunctional or dying neurons accumulate. The resulting loss of inhibitory neuronal ligands and increase in neurodegenerative components leads to activation of microglia, indicated by altered morphology, increased proliferation and upregulation of cell surface molecules. Perivascular macrophage numbers also increase. As illustrated in Panel d, in the setting of chronic neurodegeneration, the effect of a systemic inflammatory stimulus on the CNS is amplified owing to increased numbers of perivascular macrophages and microglia, as well as the primed state of microglia. The resulting exaggerated release of cytokines and other inflammatory mediators leads to neurotoxicity.

### (I-C) Immune Cells and Cytokines/Chemokines: Neurometabolic Diseases

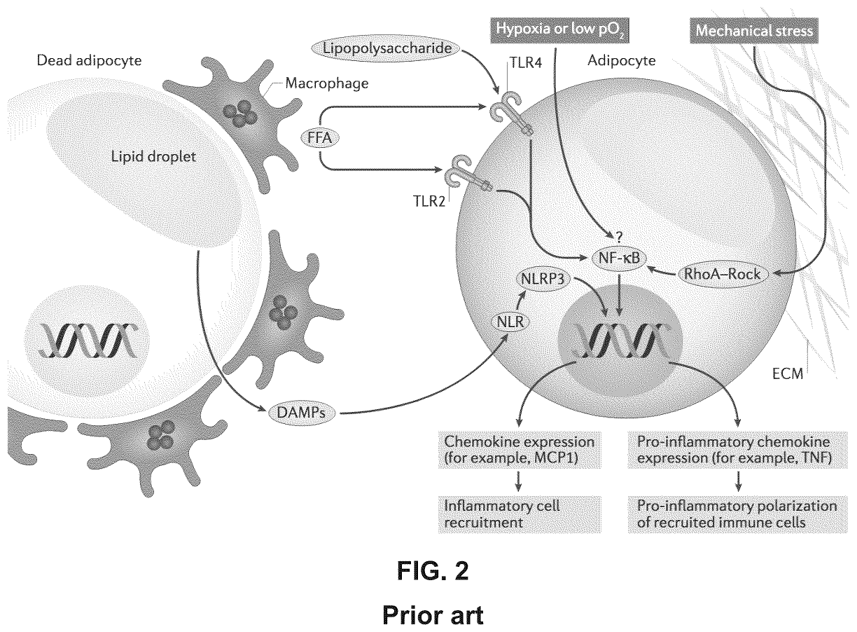

Like other neuroinflammatory disorders, obesity and diabetes have a neurological and genetic component that can cause and/or perpetuate the disease state. Obesity is defined by low-grade chronic inflammation in metabolic tissues (adipose cells, liver, brain, and pancreas), which directly contributes to insulin resistance, metabolic syndrome, and diabetes (Ellulu et al., 2017) and can be a precursor to weight gain. Adipose tissue is dysregulated in obesity and plays a central role in driving systemic inflammation and neurological comorbidities. Populations of local and infiltrating immune cells respond to inflammatory triggers, which include (1) gut-derived substances (e.g. lipopolysaccharides) that stimulate an inflammatory cascade in adipocytes via pattern recognition receptors (e.g. Toll like receptor 4; TLR4); (2) lipid species that bind to TLRs and promote downstream NFκB signaling, which can increase the synthesis and secretion of chemokines and subsequent infiltration of macrophages; (3) dying or stressed adipocytes and their damage associated molecular patterns (Jin & Flavell, 2013) and (4) mechanical stress between growing adipocytes that accumulate excessive macronutrients and the collagen-rich extracellular matrix (ECM) (FIG. 2, from Saad et al., 2016).

In a healthy state, adipocytes and immune cells coordinate energy storage and utilization. Master regulators such as IL-33 coordinate tissue integrity and metabolism via activation of lymphoid cells (via IL-33) and eosinophils (via IL-5 and IL-13) (Reilly & Saltiel, 2017). Eosinophils secrete IL-4, which maintains macrophages in their M2 (anti-inflammatory) state. In turn, M2 cellular pathways induce expression of genes that encode anti-inflammatory proteins including arginase and IL-10, which preserve insulin sensitivity and downstream metabolic flexibility (Fujisaka et al., 2009). In obesity, molecular signals are aimed at preserving tissue integrity in the context of persistent overnutrition, which includes the master regulator tumor necrosis factor (TNF) and an influx of M1 (pro-inflammatory) macrophages, which increases the M½ macrophage ratio and additional secretion of TNF and IL 1β. Likewise, other overactive resident immune cells like T cells, B cells, and dendritic cells contribute to the ongoing hyper-immune response and progression of chronic neuroinflammatory disease (Lu et al., 2019), but also provide an opportunity for novel interventions that aim to restore tissue homeostasis and metabolic function.

As illustrated in FIG. 2, gut-derived lipopolysaccharide could stimulate inflammatory pathways by binding to the pattern recognition receptor Toll-like receptor 4 (TLR4) at the plasma membrane. Similarly, free fatty acids (FFAs) can activate inflammatory signaling through either TLR4 or TLR2. Additionally, NLRP3 activation by damage-associated molecular proteins (DAMPs), which are released from dying adipocytes and recognized by NOD-like receptors (NLRs), could also be an important initiating step in inflammation. Hypoxic conditions are also associated with inflammation in adipocytes. Finally, mechanical stress caused by adipose tissue expansion through the extracellular matrix (ECM) is sensed by the RhoA-Rock pathway, which leads to downstream inflammatory signaling. NF-βB is a signaling hub that has been suggested to be involved in inflammatory signaling downstream of all these diverse potential initiators of adipocyte inflammation in individuals with obesity. Expression of genes that encode downstream inflammatory proteins leads to expression of adipokines, including inflammatory cytokines that promote the recruitment and activation of pro-inflammatory immune cells, such as TNF or tumor necrosis factor.

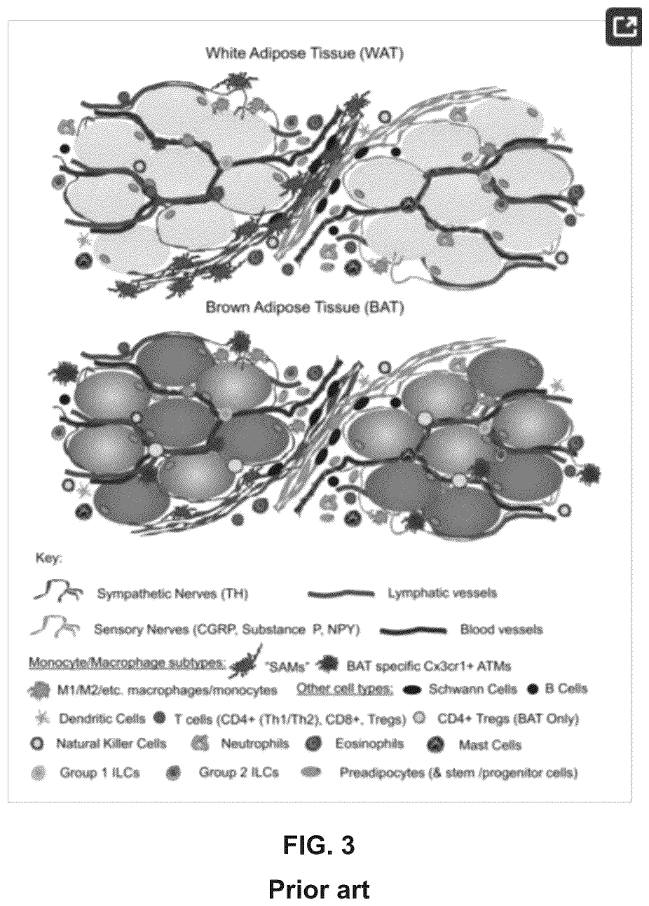

Neuroimmune communication occurs at the interface between nerve endings and their target metabolic organs (FIG. 3). In addition to cytokines and chemokines, peripheral immune cells (including macrophages in adipose tissue), synthesize and release molecules classically identified as neurotransmitters, including acetylcholine and catecholamines. Likewise, macrophages of the Cx3crl+ lineage express norepinephrine (NE) machinery, including solute carrier family 6 member 2 (Slc6a2; a known NE transporter), as well as monoamine oxidase A (MAOA), an enzyme that degrades NE (Blaszkiewicz et al., 2019). Much neuroimmune signaling occurs as a neural reflex, with signals from neural afferents (stimulated by cytokines) automatically triggering an efferent/descending signal that suppresses excessive inflammation (e.g. via vagal efferent activation of the splenic nerve) (Pavlov and Tracy, 2017). In obesity, this dynamic exchange is disrupted.

As illustrated in FIG. 3, both white adipose tissue (WAT) and brown adipose tissue (BAT) are comprised of lipid-laden mature adipocytes, where white adipocytes have one large (or unilocular) lipid droplet and brown adipocytes have many small (or multilocular) lipid droplets. The stromal vascular fraction (SVF) is the non-adipocyte cell fraction of the tissue and contains preadipocytes (and stem/progenitor cells that will undergo white or brown adipogenesis) and a milieu of immune cells. Immune cell populations include innate immune cells, such as several subtypes of monocyte-macrophages, dendritic cells, mast cells, neutrophils, eosinophils, and innate lymphatic cells (ILCs); as well as adaptive immune cells, such as several subsets of T cells, natural killer cells, and B cells. The neurovasculature of WAT and BAT includes blood vessels, lymphatic vessels, and a dense nerve supply of both sensory and sympathetic fiber types, although it is currently unclear if one tissue has a greater extent of innervation than another, or if their nerve plasticity (such as with cold or exercise) differs between tissues or depots. Some nerves innervate the vasculature itself, while other nerves innervate the parenchyma of the tissue. It is currently unclear which cell types are directly innervated and receive synaptic input.

### (II) Metabolic Signals

Nutritional science and probiotics have gained notoriety in preventive and holistic medicine, shedding light on the so-called ‘brain-gut axis,’ which mediates communication between the central nervous system (CNS) and the gastrointestinal system (GI). The vagus nerve is a major component of this bidirectional cellular communication highway, overseeing a vast array of important bodily functions including but not limited to immune response, heart rate, mood control, digestion, etc. The most important function of the vagus nerve is afferent, bringing information of the inner organs, such as gut, liver, heart, and lungs to the brain. This suggests that the inner organs are major sources of sensory information to the brain and connect emotional and cognitive areas of the brain with gut functions (Breit, 2018). The gastrointestinal nervous system/enteric nervous system (ENS) controls GI behavior independently of the central nervous system (CNS). Many neurotransmitters, signaling pathways and anatomical properties are common to the ENS and CNS, and therefore, many CNS diseases have GI symptoms. Chronic disorders with both GI and CNS consequences include Parkinson's disease, Alzheimer's disease, amyotrophic lateral sclerosis (ALS), and autism spectrum disorder (ASD), to name a few. Most interesting is that proteins and hormones released by the ENS into the blood stream can cross the blood-brain barrier (BBB) as well as interact with the vagus nerve and regulate food intake and appetite (Hagemann, 2003).

### (II-A) Metabolic Signals: Neurodevelopmental Diseases

ASD is considered a neurodevelopmental disorder, but autistic children often present with metabolic comorbidities and gut disorders, suggesting a systems-level change in metabolic signaling. For example, children with ASD can present with impaired energy metabolism and mitochondrial dysfunction, showing abnormal levels of ATP, pyruvate, creatine kinase, complex 1, caspase 7, and lactate dehydrogenase, alongside lower serum NAD+ (El-Ansary et al., 2017; Khemakhem et al., 2017). Likewise, antioxidant systems in ASD can be disrupted, with measurable depletion of plasmatic glutathione in its reduced form (GSH), increased ratio of oxidized/reduced glutathione (GSSG/GSH), and abnormal activity of glutathione peroxidase (GPx), catalase, and superoxide dismutase (SOD) (Table 3).

Gastrointestinal (GI) disorders are four-fold more common in children with ASD and fragile X syndrome than in neurotypicals, with constipation and diarrhea the most prevalent (McElhanon et al., 2014). In ASD, GI problems are strongly associated with worsening of behavioral symptoms, including anxiety, sensory over-responsivity, rigid-compulsive behaviors, and internalized symptoms (Marler et al., 2017; Mazurek et al., 2013; Ferguson et al., 2019), revealing an unmet medical need and potential for effective intervention. Given that the GI tract is highly dependent on glutathione and mitochondria to work efficiently, ASD patients may present with GI symptoms arising from decreased cellular energetic balance and deficiency in mitochondrial energy reserves. In turn, these disruptions can impact cognitive and language development, as well as energy metabolism.

TABLE 3
Changes in biomarkers of energy and antioxidant
status in ASD (El-Ansary et al., 2017)

| | | | | Percent | P |
|---|---|---|---|---|---|
| Parameter | Group | N | Mean ± S.D. | Change | value |
| Creatine kinase (U/L) | Control | 18 | 132.17 ± 50.42 | 62.74% ↑ | 0.011 |
| | ASD | 14 | 215.10 ± 100.64 | | |
| Ectonucleotidase | Control | 21 | 0.045 ± 0.022 | 44.12% ↑ | 0.003 |
| (ATPase) | ASD | 22 | 0.065 ± 0.020 | | |
| (μmol/min/ml) | | | | | |
| Ectonucleotidase | Control | 24 | 0.074 ± 0.035 | 32.51% ↑ | 0.014 |
| (ADPase) | ASD | 23 | 0.098 ± 0.029 | | |
| (μmol/min/ml) | | | | | |
| Na⁺/K⁺ (ATPase) | Control | 23 | 0.005 ± 0.002 | 226.69% ↑ | 0.001 |
| (μmol/min/ml) | ASD | 22 | 0.016 ± 0.005 | | |
| Inorganic phosphate | Control | 19 | 3.59 ± 1.19 | 27.74% ↓ | 0.005 |
| (μmol/ml) | ASD | 22 | 2.59 ± 0.96 | | |
| Lactate (mmol/L) | Control | 14 | 0.82 ± 0.27 | 61.97% ↑ | 0.016 |
| | ASD | 13 | 1.33 ± 0.63 | | |
| Adenosine triphosphate | Control | 18 | 2.37 ± 0.79 | 22.05 ↓ | 0.031 |
| (ATP) (μmol/ml) | ASD | 23 | 1.85 ± 0.70 | | |
| Adenosine diphosphate | Control | 16 | 0.89 ± 0.41 | 18.22% ↓ | 0.266 |
| (ADP) (μmol/ml) | ASD | 22 | 0.73 ± 0.46 | | |
| Adenosine | Control | 18 | 0.267 ± 0.129 | 15.85% ↓ | 0.329 |
| monophosphate | ASD | 23 | 0.225 ± 0.142 | | |
| (AMP) (μmol/ml) | | | | | |
| Adenylate energy charge | Control | 19 | 0.81 ± 0.08 | 1.00% ↑ | 0.760 |
| | ASD | 30 | 0.81 ± 0.09 | | |
| ADP/ATP | Control | 18 | 0.407 ± 0.228 | 7.40% ↓ | 0.665 |
| | ASD | 23 | 0.376 ± 0.211 | | |
| Catalase (U/dl) | Control | 28 | 39.43 ± 9.10 | 7.94% ↓ | 0.204 |
| | ASD | 29 | 36.30 ± 9.26 | | |
| Glutathione peroxidase | Control | 27 | 144.56 ± 49.19 | 81.67%↑ | 0.001 |
| (U/dl) | ASD | 27 | 262.62 ± 92.48 | | |
| Superoxide dismutase | Control | 25 | 1.153 ± 0.221 | 33.52% ↑ | 0.001 |
| (U/dl) | ASD | 24 | 1.540 ± 0.441 | | |
| Glutathione | Control | 27 | 33.24 ± 10.24 | 36.70% ↓ | 0.001 |
| (μg/ml) | ASD | 27 | 21.04 ± 6.86 | | |
| Lipid peroxides | Control | 27 | 10.21 ± 3.34 | 58.60% ↑ | 0.001 |
| (μmol/ml) | ASD | 27 | 16.19 ± 4.34 | | |
| Vitamin C (oxidized) | Control | 28 | 7.77 ± 1.63 | 18.00% ↓ | 0.003 |
| (mg/dl) | ASD | 24 | 6.37 ± 1.61 | | |
| Vitamin C (reduced) | Control | 28 | 2.80 ± 0.83 | 7.36% ↑ | 0.401 |
| (mg/dl) | ASD | 24 | 3.01 ± 0.92 | | |
| Vitamin C (total) | Control | 29 | 10.79 ± 2.09 | 0.07% ↓ | 0.990 |
| (mg/dl) | ASD | 28 | 10.78 ± 2.58 | | |
| Vitamin E (mg/dl) | Control | 28 | 1.93 ± 0.40 | 65.42% ↓ | 0.001 |
| | ASD | 20 | 0.67 ± 0.26 | | |

### (II-B) Metabolic Signals: Neurodegenerative Diseases

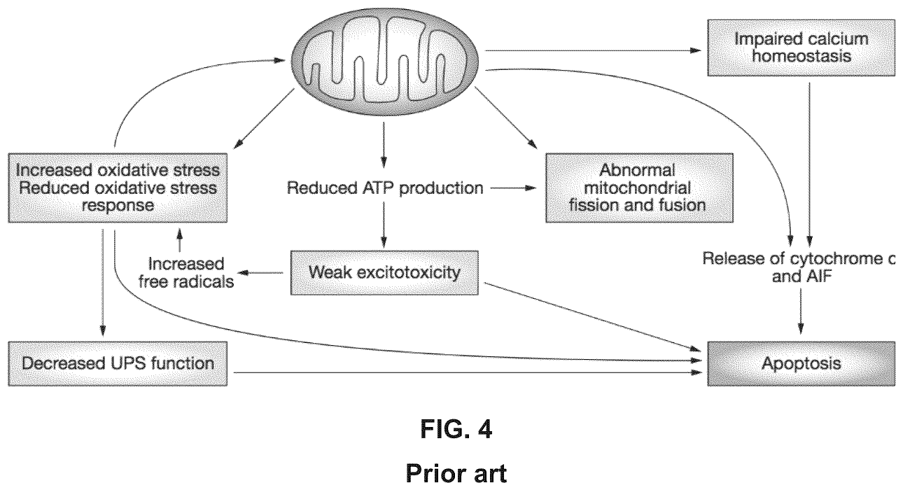

Neurodegenerative disorders such as Alzheimer's or Parkinson's disease progress through several stages over time (Johnson et al., 2018), with changes in biomarkers representing multiple physiological and pathological processes across various cell types and organs. As in autism, mitochondrial dysfunction is a key metabolic feature in Parkinson's disease. Inherited genetic mutations in elements of the electron transport chain have been implicated in parkinsonism and can perpetuate neuronal death (Henchcliffe and Beal, 2008; FIG. 4). These include parkin, α-synuclein, PINK 1, DJ-1, LRRK2 and HTRA2-all encoded by nuclear genes and all are present in the mitochondria.

Impaired metabolic processes in the brain have long been causally linked to the development of dementia (Hoyer et al., 1988) and type 2 diabetes is considered a risk factor for AD and PD (Sergi et al., 2019). Insulin has many functions in the body, including cell growth and repair, mitochondrial activity and energy utilization, autophagy and protein synthesis. Insulin can be synthesized locally in the choroid plexus or in the periphery, crossing the blood-brain barrier to target receptors on neurons. In AD and PD, insulin signaling is desensitized (Holscher, 2020): inactivation of receptors for insulin and insulin-like growth factor (IGF1) affects downstream messenger cascades that involve phosphoinositide 3 kinase (PI3 k), protein kinase B Akt/PKB, peroxisome proliferator-activated receptor (PPAR)γ/δ, and mammalian target or rapamycin (mTOR). Decreased activity of these pathways disrupts many essential functions, including cellular metabolism and resistance to oxidative stress, synaptic function and neurotransmission, and cellular repair mechanisms, which in turn perpetuates the neurodegenerative cycle.

### (II-C) Metabolic Signals: Neurometabolic Diseases

Regulation of whole-body energy homeostasis and body weight relies on an intricate balance between energy intake and expenditure, achieved by neural and hormonal controls deployed in the gut-brain axis.

The brain, especially the hypothalamus, plays a key role in the control of food intake by sensing metabolic signals from peripheral organs and modulating feeding behaviors. To accomplish these important roles, the hypothalamus communicates with other brain areas such as the brainstem and reward-related limbic pathways. The adipocyte-derived hormone leptin and pancreatic β-cell-derived insulin inform adiposity to the hypothalamus. Gut hormones such as cholecystokinin, peptide YY, pancreatic polypeptide, glucagon-like peptide 1, and oxyntomodulin transfer satiety signals to the brain and ghrelin relays hunger signals.

The hormones ghrelin and leptin communicate information to the central nervous system (CNS) about the current levels of energy reserves and nutritional status.

Ghrelin is a hormone that is produced and released mainly by the stomach with small amounts also released by the small intestine, pancreas and brain. Ghrelin has numerous functions. It is termed the ‘hunger hormone’ because it stimulates appetite, increases food intake and promotes fat storage. However, ghrelin is more than a hunger hormone because it also has the following effects:
activates its receptor, growth hormone secretagogue receptor (GHS-R)
stimulatory effects on food intake, fat deposition and growth hormone release
regulates glucose hemostasis by inhibiting insulin secretion and regulating gluconeogenesis/glycogenolysis.
decreases thermogenesis to regulate energy expenditure.
improves the survival prognosis of myocardial infarction by reducing sympathetic nerve activity.
prevents muscle atrophy by inducing muscle differentiation and fusion.
regulates bone formation and metabolism by modulating proliferation and differentiation of osteoblasts.
involved in cancer development and metastasis; ghrelin and GHS-R mRNA are highly expressed in metastatic forms of cancers.

Ghrelin signaling has increasingly been recognized as a key regulator of obesity, insulin resistance and diabetes; intriguingly, many of these regulatory functions appear to be independent of ghrelin's effect on food intake, since development of resistance to leptin and ghrelin, hormones that are crucial for the neuroendocrine control of energy homeostasis, is a hallmark of obesity.

Leptin is a 16 kDa cytokine that is produced predominantly by adipose tissue; it is released into the bloodstream and circulates in proportion to body fat mass. Consequently, levels of leptin convey information about the energy reserves of the body to the centers that regulate energy homeostasis. In obesity, resistance may develop to the anorexigenic signals (anorexigenic gastrointestinal peptides, leptin, insulin), while in calorie-restricted states no such resistance was demonstrated (as if promoting further obesity or maintenance of lean shape, respectively) (deLartigue et al., 2011).

Cholecytokinin (CCK) and ghrelin serve antagonistic functions in regulating glucose homeostasis and energy expenditure: ghrelin is orexigenic and inhibits insulin secretion, while CCK is anorexigenic, stimulating insulin secretion.

CCK-1 and ghrelin receptors are colocalized on vagal afferent neurons where they are in antagonistic interaction.

Vagal afferent neurons exhibit abnormal sensitivity to GI peptides or mechanical stimuli in pathological conditions such as diet-induced obesity or diabetes (Daly et al., 2011; Kentish et al., 2012). Both high fat and high carbohydrate diets impair vagus nerve signaling of satiety (Loper et al, 2021).

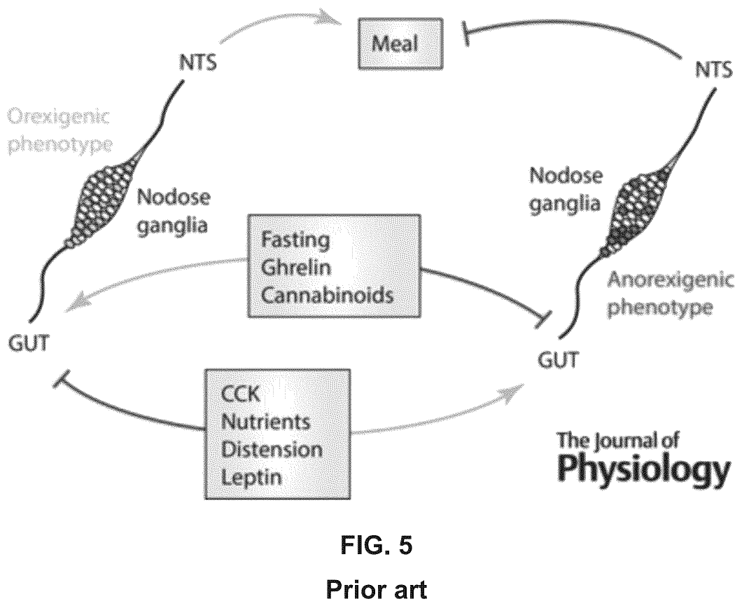

Approximately 40% of vagal afferent neurons innervate the gut. As illustrated in FIG. 5 (from de Lartigue, 2016), these neurons express two different neurochemical phenotypes that reflect the nutrient status. In response to nutrients, there is distension of the stomach and release of the satiating hormone cholecystokinin. Circulating leptin enhances the sensitivity of vagal afferent neurons to these peripheral signals, promotes vagal afferent neuron expression of receptors and neuropeptides associated with inhibiting food intake (anorexigenic phenotype—red neurons), and inhibits expression of receptors and neuropeptides associated with stimulating food intake (orexigenic phenotype—green neurons). In the absence of food, ghrelin and cannabinoids are released and inhibit expression of the anorexigenic phenotype in preference for the orexigenic phenotype in vagal afferent neurons. Release of anorexigenic neuropeptides from vagal afferent neurons to the NTS shortens the duration of meals and reduces their size, while the release of orexigenic neuropeptides prolongs meals and increases their size.

Another important hormone and signaling molecule is Glucagon-like peptide 1 (GLP-1), which is produced in the gut and in the brain. This hormone controls neural activity in brain regions regulating food intake, where increased GLP-1 is a satiety signal.

### (III) Serotonergic Signaling

The serotonergic system plays a tonic modulatory role during brain development and interacts with cellular cascades involved in synaptic maturation and brain plasticity. During normal brain development, serotonin signaling plays a tonic modulatory role, affecting many systems in the body via a widespread axonal network (Wirth et al., 2017).

### (III-A) Serotonergic Signaling: Neurodevelopmental Diseases

Reflecting its dominating neurodevelopmental control, serotonin levels peak in neurotypical children between ages 2 and 5 years, then gradually decrease to reach adult levels by approximately age 15—a difference of >200%. In contrast, serotonin levels are much lower in children with developmental delay (Chugani, 2002) and increase gradually, missing the peak of early childhood. As serotonin is also involved in synaptic remodeling and maturation (Bijata et al., 2017), serotonergic insufficiency may have a compounding effect in neurodevelopmental disorders manifesting as behavioral and emotional symptoms like cognitive rigidity, learning disability, aggression, exaggerated fear response, and/or depression (Lesch et al., 2012). Likewise, activity-dependent cellular pathways that underlie circuit plasticity are affected (Bagni and Zukin, 2019), in effect decoupling developmental brain changes from neural stimulation-driven signal optimization. Thus, children with autism rely less on input from their environment and more on existing neural frameworks, which can present clinically as inattention, hyperarousal, and hypersensitivity, though many more variations are possible. Such diversity in symptomatology makes autism difficult to treat and no effective medicines currently exist.

### (III-B) Serotonergic Signaling: Neurodegenerative Diseases

Several lines of evidence point to the role of serotonin signaling in neurodegenerative diseases.
Serotonin 2A receptor autoantibodies increase in adult traumatic brain injury in association with neurodegeneration (Zimering et al., 2020).
Type 2 diabetes and traumatic brain injury (TBI) are associated with an increased risk of late-onset neurodegeneration via mechanisms involving increased peripheral and central inflammation, respectively.
In older adult type 2 diabetic subsets having Parkinson's disease or dementia, circulating plasma immunoglobulin G (IgG) autoantibodies bind to the 5-hydroxytryptamine 2A receptor and mediates neurotoxicity in mouse neuroblastoma cells through activation of Gq11/inositol triphosphate receptor (IP3R)/Ca2+ and RhoA/Rho kinase signaling pathways.
The 5HT2A receptor is highly concentrated in brain regions underlying cognition, memory, perception, and mood regulation [9]. Increased circulating 5-HT2AR IgG autoantibodies in traumatic brain injury might provide a biomarker (or be involved in the pathophysiology) of the later occurrence of neurodegenerative complications.

There is neurobiological and clinical continuum between depression and Alzheimer's disease (AD) (Caraci et al., 2018). Depression is a risk factor for the development of AD, and the presence of depressive symptoms significantly increases the conversion of Mild Cognitive Impairment (MCI) into AD. Common pathophysiological events have been identified in depression and AD, including neuroinflammation with an aberrant Tumor Necrosis Factor-a (TNF-α) signaling, and an impairment of Brain-Derived Neurotrophic Factor (BDNF) and Transforming-Growth-Factor-β1 (TGF-β1) signaling.

TGF-β1 is an anti-inflammatory cytokine that exerts neuroprotective effects against amyloid-β (Aβ)-induced neurodegeneration, and it has a key role in memory formation and synaptic plasticity. TGF-β1 plasma levels are reduced in major depressed patients (MDD), correlate with depression severity, and significantly contribute to treatment resistance in MDD. The deficit of Smad-dependent TGF-β1 signaling is also an early event in AD pathogenesis, which contributes to inflammation and cognitive decline in AD. A long-term treatment with antidepressants such as selective-serotonin-reuptake inhibitors (SSRIs) is known to reduce the risk of AD in patients with depression and, SSRIs, such as fluoxetine, increase the release of TGF-β1 from astrocytes and exert relevant neuroprotective effects in experimental models of AD.

### (III-C) Serotonergic Signaling: Neurometabolic Diseases

In the periphery, brain-gut interactions surrounding satiety are mediated in part by the vagus nerve— a bi-directional neural pathway responsible for transmitting information about hunger, discomfort, and fullness. For example, vagal afferent neurons express receptors for both cholecystokinin (CCK) and ghrelin, which serve antagonistic functions in regulating glucose homeostasis and energy expenditure: ghrelin is orexigenic and inhibits insulin secretion, while CCK is anorexigenic, stimulating insulin secretion.

In the gastrointestinal tract, locally synthesized serotonin promotes gut peristalsis and nutrient absorption, and upon entering the bloodstream, facilitates insulin secretion and de novo lipogenesis (Yabut et al., 2019). In the central nervous system, serotonin regulates mood and behavior, suppresses appetite, and promotes energy utilization (Tecott, 2007). For example, 5-HT2C receptors are expressed in the arcuate nucleus (ARC) of the hypothalamus (the brain center of hunger and satiety). Here, serotonin receptors play a central role in sensing peripheral metabolic signals of nutritional status (Oh et al., 2016), including signaling overlap with the appetite-suppressing leptin and insulin. Also, serotonin signaling is involved in cellular and behavioral decision-making on food intake and energy expenditure (Yu and Kim, 2012). One example is the serotonergic agonist fenfluramine, which was shown to activate 5-HT2C receptor to induce anorexia (Heisler et al., 2002). Thus, serotonin (5-HT) modulates energy homeostasis.

Several 5-HT receptors have been implicated in the regulation of metabolic homeostasis, including 5-HT1B, 5-HT1F, the 5-HT2 receptors (5-HT2A-C), 5-HT3 and 5-HT6 (Table 4, from Wyler et al., 2017) (See also Namkung et al., 2015; Voigt and Fink, 2015).

TABLE 4
from Wyler et al., 2017

| Receptor | Signaling pathway | Tissue expression | Function |
|---|---|---|---|
| 5-HT_{1B} | G [symbol] | AgRP/NPV neurons | Suppress food intake |
| 5-HT [symbol] | G [symbol] | β-cell (pancreas) | Inhibits β-cell proliferation |
| 5-HT_{1F} | G [symbol] | α-cell (pancreas) | Inhibits glucagon secretion |
| 5-HT_{2A} | G [symbol] | White adipose | Inhibits lipolysis |
| | | | Promotes lipogenesis |
| 5-HT_{2B} | G [symbol] | White adipose | Promotes lipolysis |
| | | β-cell (pancrease) | Promotes β-cell proliferation |
| | | | Promotes insulin secretion |
| | | Liver | Promotes gluconeogenesis |
| | | | Inhibits hepatic glucose uptake |
| 5-HT [symbol] | G [symbol] | PCMC/CART neurons | Suppress food intake |
| 5-HT [symbol] | Cation channel | Brown adipose | Suppress BAT thermogenesis |
| | | β-cell (pancrease) | Promotes insulin secretion |
| 5-HT [symbol] | G_{N} | PVN | Promotes food intake |
| 5-HT_{T} | | IML | Modulates sympathetic outflow? |

Abbreviations:
PVN, paraventriclar nucleus;
IML, intermediolateral nucleus. [symbol]
indicates data missing or illegible when filed

The inhibition of central 5-HT synthesis with the TPH inhibitor, para-chlorophenylalanine (pCPA) or chemical lesion of 5-HT neurons with 5,7-dihydroxytryptamine (5,7-DHT) is orexigenic (Breisch et al., 1976; Saller and Stricker, 1976). Conversely, increasing synaptic 5-HT bioavailability, either by facilitating vesicular release with fenfluramine or by inhibiting 5-HT reuptake with selective serotonin reuptake inhibitors (SSRIs), produces an anorexigenic effect (Simansky and Vaidya, 1990; Heisler et al., 1997; Heal et al., 1998; Silverstein-Metzler et al., 2016). In addition to reducing food intake, increased central 5-HT signaling also improves glucose homeostasis, as treatment with fenfluramine or meta-chlorophenylpiperazine (mCPP, a 5-HT1B/2C receptor agonist) improves glucose tolerance and insulin sensitivity (Storlien et al., 1989; Zhou et al., 2007).

Based in part on the above discussion, Table 5 shows how levels of each of the mRNAs would increase or decrease with the presentation of each disease or disorder, or, conversely upon effective treatment of each disease or disorder.

TABLE 5
Effect of various diseases on specific mRNAs.

| | ASD | FXS | AD | PD | Obesity | Diabetes |
|---|---|---|---|---|---|---|
| Cytokines | | | | | | |
| eotaxin-1 (CCL11) | up | down | up | up | up | up |
| eotaxin-2 (CCL24) | up | CCL22 down | up | up | up | up |
| eotaxin-3 (CCL26) | up | down | up | up | up | up |
| IL-5 | up | up | down | up | | up |
| MCP-1 (CCL2) | up | down (FXS + AU) | up | up | up | up |
| MIP-1a (CCL3) | up | down | up | no change | no change | up |
| GROa (CXCL1) | up | CXCL10 down | up | up | CXCL2 & 5 up | CXCL2 & 5 up |
| RANTES (CCL5) | down | down | up | up | up | up |
| MCP-3 (CCL7) | down | down | up | up | up | up |
| IL-1B | up | up | up | up | up | up |
| IL-6 | up | no change | up | up | up | up |
| IL-8 | up | no change | up | up | up | up |
| IL-12A | up | up (FXS + AU) | up | up | up | up |
| TGFB | down | down | up | up | up | up |
| TNF-alpha | up | no change | up | up | up | up |
| IL-4 | up | no change | up | up | up | up |
| IL-13 | up | no change | down | up | up | up |
| IL-10 | down | down | down | down | up | up |
| Cytokine targets | | | | | | |
| CCR3 | up | up | CCR2 up | CCR2 up | up | CCRS up |
| NOD1 | up | up | up | up | up | up |
| NLRA | NLRP3 up | NLRP3 up | NLRP3 up | NLRP3 up | NLRP12 down | NLRP12 down |
| IFNGR | up | no change | up | up | up | up |
| HAAO/IDO | down | down | up | up | up | up |
| Neurotransmission | | | | | | |
| NMDAR1 (GRIN1) | up | up | up | up | up | up |
| GRIN2B | up | up | up | up | up | up |
| GRIN3A | up | up | up | up | up | up |
| GRIN2A | up | up | up | up | up | up |
| TLR-4 | up | up | up | up | up | up |
| TLR3 | up | up | up | up | up | up |
| TLR1 | up | up | up | up | up | up |
| MAOA | down | down | up | up | down | down |
| Serotonin Signaling | | | | | | |
| TPH1 | age-dependent | down | down | down | up | regional |
| TPH2 | age-dependent | down | down | down | down | down |
| AADAT/KAT2 | down | down | up | up | up | up |
| Membrane proteins/channels | | | | | | |
| OCTN1 | up | up | up | up | up | up |
| P2X4 | down | P2 × 3 up | up | up | up | up |
| P2X7 | down | no change | up | up | up | down |
| VDAC3 | VDAC down | VDAC1 down | up | up | VDAC1 up | VDAC1 up |
| SLC22A15 | up | up | up | up | up | SLC25A35 up |
| SLC22A3 | up | up | up | up | up | up |
| SLC3A2 | SLC30A3 up | up | up | up | SLC30A3 up | SLC30A3 up |
| DNA damage repair | | | | | | |
| ZNF365 | up | up | up | up | up | up |
| TRIM26 | TRIM27 up | TRIM27 up | TRIM11 down | TRIM11 down | up | up |
| ZNF827 | up | up | up | up | up | up |
| Neurotrophic/growth factors | | | | | | |
| GDNF | no change | down | down | down | down | down |
| PDGF-A | down | up | down then up | down | up | PDGFRB up |
| NGF | no change | down | down | down | up | down |
| FGF-13 | down | down | FGF14 down | FGF14 down | FGF21 up | FGF19 down |
| GIF/MT-III | down | down | down | up | up | up |
| Other | | | | | | |
| A2AP | no change | no change | up | up | up | up |
| CAPG | down | down | up | up | up | up |
| HMBS/PBGD | down | down | down | down | down | no change |

Additionally provided is a method of developing a treatment of a disease or disorder in a patient, where the disease or disorder is a neurodevelopmental disease, a neurodegenerative disease, or a neurometabolic disease. The method comprises

treating more than one patient having the disease or disorder with the treatment, and

determining mRNA levels in the patient of one or more genes for an inflammatory cytokine, a cytokine target, neurotransmission, serotonin signaling, a membrane channel, DNA damage repair, a growth factor, A2AP, CAPG, and/or NHBS, wherein

if the mRNA levels indicate that the treatment alleviates a symptom of the disease or disorder, development of the treatment is continued, and

if the mRNA levels indicate that the treatment does not alleviate a symptom of the disease or disorder, development of the treatment is discontinued.

The mRNA levels evaluated in this method are as previously discussed.

Development of any treatment for the disease or disorder can utilize this method. In some embodiment, the treatment is administration of a medication.

Administration of any medication, by any route, as previously discussed, can utilize this method. In some embodiments, the medication is a psychoactive tryptamine derivative.

Administration of any psychoactive tryptamine derivative, as previously discussed, can be developed using this method. In some embodiments, the psychoactive tryptamine derivative is psilocybin baeocystin, aeruginascin, or psilocin.

Other aspects of this method have also previously been discussed.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1. Leveraging the Unique Language of Cellular Communication for Psychedelic Drug Development and Treatment

Diverse emotional states, behaviors, and thoughts that affect quality of life are an everyday part of chronic neuroinflammatory conditions such as Alzheimer's disease, Parkinson's disease, depression, addiction, autism spectrum disorder, and even obesity.

What cellular communication in the brain underlies such disruption in daily activities? Does the gut play a role? What is the cellular language of orchestrated biological activity? What happens when things go wrong?

### The Language Communication Playing Field:

Nerve cells communicate through their own language which involves sending and receiving signals—much like an electrical current that must be transmitted and elicit a response. Signals may be electrical or chemical and may originate within the cells or in their surrounding environment.

Signal transduction refers to the transmission of a molecular signal, in the form of a chemical modification. Protein complexes move along a signaling pathway that ultimately triggers a biochemical event. Activation of a specific receptor, either on the cell surface or inside of the cell triggers a signaling cascade that eventually elicits a response. Depending upon the cell involved, this response may alter the cells metabolism, shape, gene expression, or ability to divide.

At any step, the signal can be amplified— a single molecule can generate a response involving hundreds to millions of molecules. These molecules are often secreted by the cell and released into the extracellular space where other cells respond and react. To trigger a response, the signal must be transmitted across the cellular membrane. Other times the signal works by interacting with receptor proteins that contact both the outside and inside of the cell. In this case, only cells that have the correct receptors on their surfaces will respond to the signal.

Cellular communication is a dynamic system that adjusts itself in response to the local and systemic cellular environment. For example, the immune system uses local and systemic signal transduction mechanisms to defend the body from disease or invading pathogens. Normally, the immune response is activated and then promptly terminated to avoid excessive tissue damage. In a neuroinflammatory state, the immune response is sustained and becomes disrupted, as cells dynamically adjust their activation threshold.

A good example of a pathogen-activated immune response and the subsequent production of signals is shown by the brain's response to endotoxemia, the presence of bacteria in the blood stream (Srinivasan, K. et al. 2016).

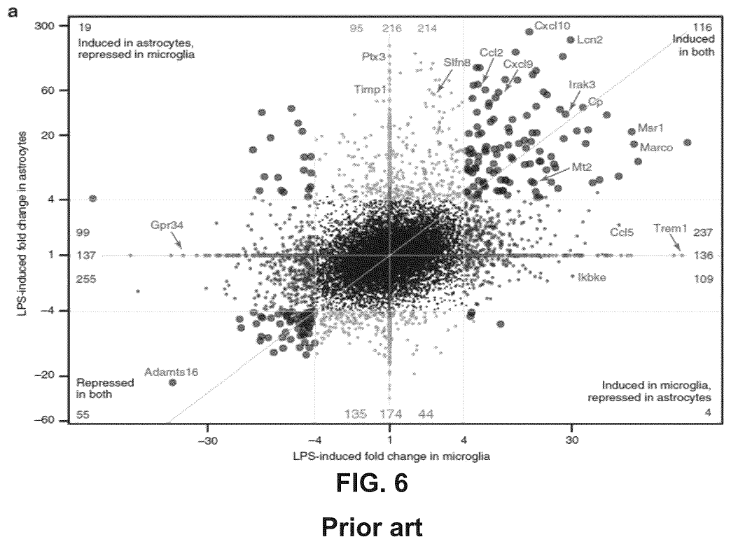

The scattergram picture (FIG. 6) shows the inflammatory genetic response within the brain of a mouse to a bacterial blood infection. The various signals show different gene expression profiles for the immune cells in the central nervous system (CNS): microglia and astrocytes, which mediate much of the neuroinflammatory response. The peripheral endotoxemia had a profound effect on gene expression in both microglia and astrocytes, as samples within these cell types clustered naturally into treatment and control groups (Srinivasan, 2016).

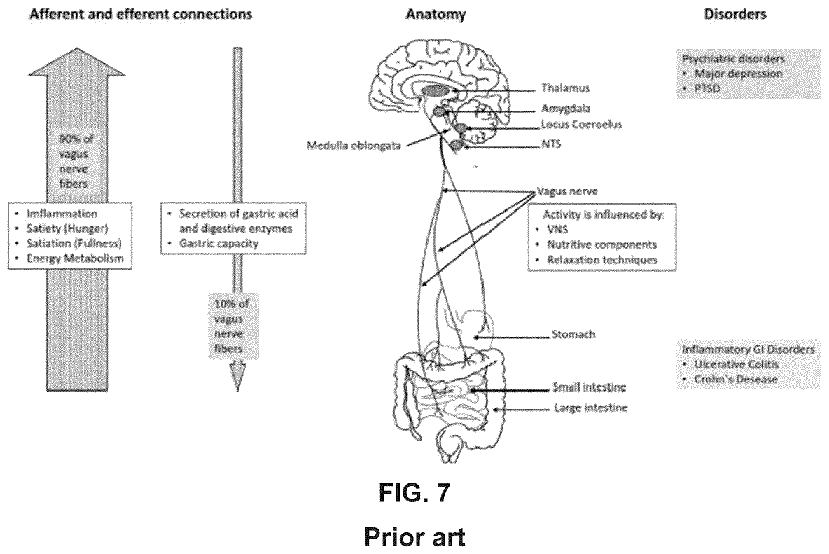

As illustrated in FIG. 7, nutritional science and probiotics have gained notoriety in preventive and holistic medicine, shedding light on the so-called ‘brain-gut axis,’ which mediates communication between the CNS and the gastrointestinal system (GI). The vagus nerve is a major component of this bidirectional cellular communication highway, overseeing a vast array of important bodily functions including but not limited to immune response, heart rate, mood control, digestion, etc. The most important function of the vagus nerve is afferent, bringing information of the inner organs, such as gut, liver, heart, and lungs to the brain. This suggests that the inner organs are major sources of sensory information to the brain and connect emotional and cognitive areas of the brain with gut functions (Breit, 2018). Thus, when the neuroinflammatory state affects the vagus nerve, symptoms appear along the entire brain-gut axis, affecting cognition, mood, and flexibility in the social environment.

The gastrointestinal nervous system/enteric nervous system (ENS) controls GI behavior independently of the central nervous system (CNS). Many neurotransmitters, signaling pathways and anatomical properties are common to the ENS and CNS, and therefore, many CNS and metabolic diseases have GI symptoms. Chronic disorders with both GI and CNS consequences include Parkinson's disease, Alzheimer's disease, amyotrophic lateral sclerosis (ALS), autism spectrum disorder (ASD), and obesity to name a few. Most interesting is that proteins and hormones released by the ENS into the blood stream can cross the blood-brain barrier (BBB) as well as interact with the vagus nerve and regulate food intake and appetite (Hagemann, 2003).

Key players in the GI tract are gut microbiota and are involved in bidirectional communication between the gut and the brain. “Based on evidence, the gut microbiota is associated with metabolic disorders such as obesity, diabetes mellitus and neuropsychiatric disorders such as schizophrenia, autistic disorders, anxiety disorders and major depressive disorders. In the past few years, neuroscientific research has shown the importance of the microbiota in the development of brain systems” (Evrensel, 2015).

An emerging role for mRNA protein coding genes; Are they hard biomarkers?

A prime example is the chemokine Eotaxin-1 (CCL11) which was originally implicated in the recruitment of eosinophiles into inflammatory sites during allergic reactions. In addition to its previous accepted role in immune response, studies have now revealed that eotaxin-1/CCL11 is associated with aging, neurogenesis, and neurodegeneration, being able to influence neural progenitor cells, and microglia. Increased circulating levels of eotaxin-1/CCL11 have been described in major psychiatric disorders (schizophrenia, bipolar disorder, major depression), sometimes correlating with the severity of psychopathological and cognitive parameters (Teixera, 2018). Another cytokine IL10 has potent anti-inflammatory properties and plays a role in limiting host response to pathogens thereby preventing damage to the host while maintaining normal tissue function. IL-10 also limits inflammation in the brain; it does so by three major pathways: (1) reducing synthesis of proinflammatory cytokines, (2) suppressing cytokine receptor expression, and (3) inhibiting receptor activation (Strle, 2001).

The next questions are whether eotaxin-1/CCL11 and IL10 can be regarded as prognostic biomarkers of disease and/or as therapeutic targets for resistant/progressive cases. “The regulation of IL-10 production by central nervous cells remains a challenging field. Answering the many remaining outstanding questions will contribute to the design of targeted approaches aiming at controlling deleterious inflammation in the brain” (Lobo-Silva, 2016).

We sought to answer these questions in a recent fragile X syndrome preclinical study using the KO knockout rat and wild type (WT) controls. We found differences in neuroinflammatory biomarkers between KO and WT animals. Moreover, we uncovered significant changes in serum cytokine levels pre and post treatment with psilocybin. Analytic cytokine flow cytometry studies were performed by Thermo Fisher Scientific, Vienna BioCenter, Austria (Perederiy and Hausman et al., manuscript in preparation).

The FDA Psychedelic Drug Chess Game:
Manufacture synthetic c-GMP psilocybin, greater than 98% purity.
Show proof of efficacy for desired therapeutic claim; Positive behavioral changes without associated cognition loss side effects.
Prepare Drug Master File (DMF) to be submitted to regulatory agency for specific phase of human IND study.
Establish data bank with hard biomarkers to confirm therapeutic behavioral changes with drug treatment. Avoid basing results solely on biased behavioral testing.
In the case of autism spectrum disorder (ASD), for example, confirm behavior changes with positive pre- and post-treatment mRNA neuroinflammatory biomarker data.

### Conclusion:

Neuroinflammatory conditions—especially those that have the added complexity of disrupted emotional states and behaviors— are still difficult to diagnose accurately and treat effectively. We need new biomarkers that characterize aberrant gene expression and cellular communication in neurological disease or injury and provide insights into possible interventions and disease monitoring.

The multiple functions of mRNA inflammatory genes in the brain, as well as in peripheral organs, will create new and intriguing vistas that will promote a better understanding of neurodegenerative diseases. These discoveries could lead to development of innovative approaches for the use of anti-inflammatory cytokines in major debilitating diseases of the CNS.

Psychedelic medicine is offering much promise as a new therapeutic paradigm with the potential to provide much needed treatment options, especially in areas with unmet medical needs. The ability to penetrate the unique genetic language underlying the development of chronic diseases and assess therapeutic responses will assist in obtaining psychedelic drug approval with the regulatory agencies.

### Example 2. Cytokine Expression Alterations in Fragile X Disease

Preclinical proof-of-concept data show that profiles of microbiota and inflammatory cytokines are statistically different in the offspring of pregnant rats treated with saline versus offspring of pregnant rats treated with valproic acid (VPA)— an established preclinical model that displays key features of autism spectrum disorder (ASD) and reflects its multifactorial etiology. In the same model, we showed that treatment with psilocybin is safe and improves anxiety-like behaviors. Further confirming the high safety profile, we found that psilocybin and psilocin are non-toxic and have a protective effect against oxidative stress in red blood cells.

### Methods

We have tested psilocybin treatment in a Fmr1 knock-out (KO) Sprague-Dawley rat model of Fragile X Syndrome (FXS) and showed that repeated microdoses of psilocybin reverted the cognitive impairment displayed by Fmr1 KO juvenile rats without affecting the performance in wild-type (WT) control animals (Example 3). Likewise, the following cytokines were significantly different between KO and WT animals:
IL-5: Model showed a significant difference in IL-5 between genotypes (F(1, 74)=4.532; p=0.037)
Eotaxin: Model showed a significant difference in Eotaxin between genotypes (F(1, 74)=5.133; p=0.026), and across Dose (F(2, 74)=5.090; p=0.008). Pairwise comparison within dosing showed significant differences between wt/veh v. wt/3 mg/kg (t(24.974)=−2.2557; p=0.033), AND KO/1 mg/kg v. KO/3 mg/kg (t(24.804)=−2.5127; p=0.019)
GROα: Model showed a significant difference in GROα between genotypes (F(1, 73)=4.336; p=0.041)
MCP-1: Model showed a significant difference in MCP-1 between genotypes (F(1, 74)=12.378; p=0.0007). We observed a near-significant effect of Dose (F(2, 74)=2.448; p=0.093), and thus explored potential pairwise differences by dose. Comparison showed significant differences between wt/veh v. wt/3 mg/kg (t(23.834)=−2.9367; p=0.0072), AND wt/1 mg/kg v. wt/3 mg/kg (t(24.424)=−2.1068; p=0.0456)
MCP-3: Simple main effects analyses indicated a significant difference between wt and KO (F(1, 78)=17.55; p=7.31e-5), as well as a near-significant effect of dose (F(2, 77)=2.597; p=0.08). Although the latter didn't achieve true significance, we examined pairwise comparisons to see what might be driving the near-effect. Pairwise comparisons revealed significant differences between wt/veh v. wt/3 mg/kg (t(24.907)=−36823; p=0.001), KO/veh v. KO/1 mg/kg (t(22.339)=2.2053; p=0.038), and KO/1 mg/kg v. KO/3 mg/kg (t(24.488)=−2.7772; p=0.01).
RANTES: Model showed a significant difference in RANTES between genotypes (F(1, 74)=9.691; p=0.003).

### Example 3. Psilocybin Mitigates the Cognitive Deficits Observed in a Rat Model of Fragile X Syndrome

### Introduction

Fragile X Syndrome (FXS) is the most common form of inherited intellectual disability (ID) and the leading monogenic cause of ASD. Thus, FXS patients show several ASD-like symptoms, including social dysfunction, hyperactivity, stereotypic movements, hand flapping, hand biting, speech delay, and a relative lack of expressive language ability. Overall, approximately 30% of patients with FXS meet the full diagnostic criteria for ASD (Harris et al., 2008), while over 90% of individuals with FXS display some ASD symptoms (Hernandez et al., 2009). FXS is an X-linked pathology, associated with the wobbling expansion of a CGG trinucleotide repeat within the 5′-untranslated region (5′UTR) of the FMR1 gene, ultimately leading to the heterochromatinization of the FMR1 locus and, as a consequence, to the absence of expression of the FMR1 gene and its related protein product, the Fragile X Mental Retardation Protein (FMRP) (Bardoni et al., 2006). FMRP is a modulator of the translation of synaptic proteins and mRNA transport at the synapse and it plays a key role in several neural pathways that are disrupted in ASD (Darnell and Klann, 2013).

Serotonin has a key role in normal brain development (Wirth et al., 2017): humans undergo a period of high brain serotonin synthesis during childhood and this developmental process is disrupted both in autism (Chugani, 2002) and FXS (Hanson and Hagerman, 2014). As serotonin is involved in synaptic remodeling and maturation, serotonergic insufficiency during childhood may have a compounding effect on brain patterning in ASD and FXS, manifesting as behavioral and emotional symptoms (Lesch and Waider, 2012). As such, compounds that stimulate serotonergic signaling such as psilocybin and other tryptamine derivatives may offer promise as effective early interventions for developmental disorders such as ASD and FXS. To support this possibility, clinical and preclinical studies have shown that psilocybin positively affects several facets of social behavior relevant to deficits in ASD (Markopoulos et al., 2021; Pokorny et al., 2017; Preller et al., 2016). Furthermore, serotonin receptor agonists improved the synaptic malfunction exhibited by FXS mice (Costa et al., 2015). Noteworthy, recent clinical trials of psilocybin in psychiatric disorders yield exciting results (Nutt et al., 2020). Early clinical studies from the 1960s and 70s assessed the use of psilocybin in the treatment of children classified as “autistic-schizophrenic”, reporting some positive effects (Markopoulos et al., 2021). However, the reported adverse effects, the small sample size of these studies and the change in current diagnostic criteria for ASD demands caution in the interpretation of these early findings.

In this scenario, the aim of the present study was to test whether different protocols of psilocybin administration mitigate the cognitive deficits displayed by the recently validated Fmr1-^{Δ}exon 8 rat model of ASD, that is also a model of FXS.

### Materials and methods

### Animals

The experiments were performed in Fmr1-^{Δ}exon 8 rats on a Sprague-Dawley background (Horizon Discovery, formerly SAGE Labs, USA), proposed as a genetic model of ASD and rat model of FXS (Golden et al., 2019; Schiavi et al., 2022); the corresponding wild-type (WT) animals were used as controls.

Pregnant rats were individually housed in Macrolon cages (40 (1)×26 (w)×20 (h) cm), under controlled conditions (temperature 20-21° C., 55-65% relative humidity, and 12/12 h light cycle with lights on at 07:00 h). Newborn litters found up to 17:00 h were considered to be born on that day (postnatal day, PND 0). On PND 1, the litters were culled to eight animals (six males and two females) to reduce any litter size-induced variability in the growth and development of pups during the postnatal period. On PND 21, the pups were weaned and housed in groups of three (same sex and genotype). One pup per litter from different litters per treatment group was randomly used in each experiment. Male offspring were tested at adolescence between PND 42 and 49. The sample size was based on our previous experiments and power analysis performed with the GPower software. Sample size (n) is indicated in the figure legends. A trained observer, who was unaware of the treatments, scored the behavioral tests using the Observer 3.0 software (Noldus, The Netherlands). the experiments were performed in agreement with the ARRIVE (Animals In Research: Reporting In Vivo Experiments) guidelines (Kilkenny et al., 2010), the guidelines of the Italian Ministry of Health (D. L. 26/14, 988/2020-PR), and the European Community Directive 2010/63/EU.

Drugs and Experimental Design

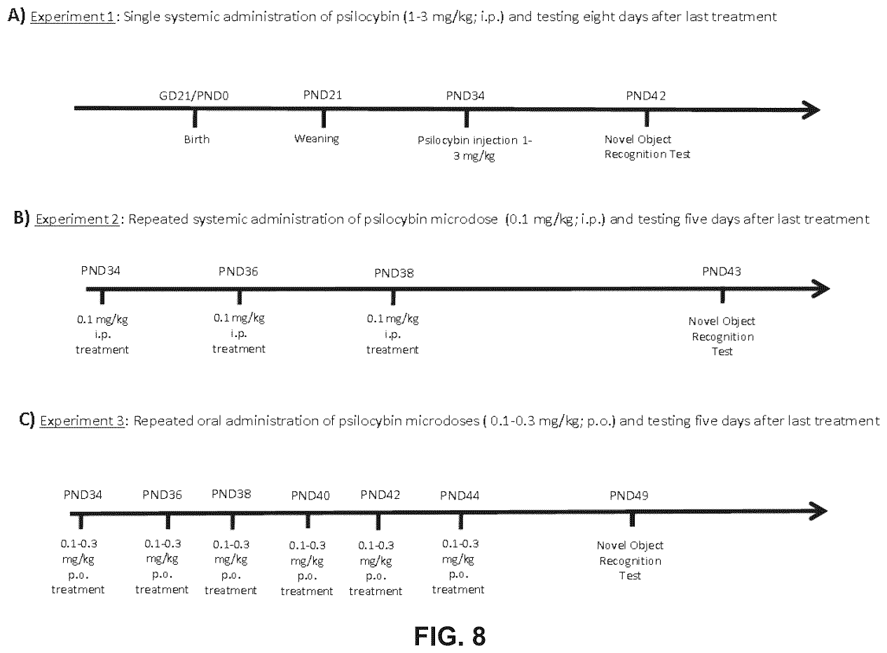

We performed three experiments testing Fmr1-^{Δ}exon 8 rats and WT controls in the novel object recognition task using three different treatment schedules for psilocybin administration:
1. Experiment 1: a single dose of either 1 (Hibicke et al., 2020) or 3 mg/kg psilocybin given intraperitoneally (i.p.) eight days before behavioral testing (FIG. 8A);
2. Experiment 2: repeated microdoses of psilocybin (0.1 mg/kg) given i.p. three times every other day, with behavioral testing starting five days after the last administration (FIG. 8B);
3. Experiment 3: repeated microdoses of psilocybin (0.1 or 0.3 mg/kg) (Higgins et al., 2021) given orally (p.o.) three times every other day, for two consecutive weeks, with behavioral testing starting five days after the last administration (FIG. 8C).

Psilocybin was dissolved in saline. Solutions were freshly prepared before treatment.

### Behavioral Tests

### Novel Object Recognition

We have recently shown that Fmr1-^{Δ}exon 8 rats show impaired novel object recognition memory at adolescence (Schiavi et al., 2022). We here tested the ability of psilocybin to mitigate the cognitive deficit displayed by adolescent Fmr1-^{Δ}exon 8 rats in this task. On the training trial, each rat was individually placed into an open-field arena containing two identical objects (A1 and A2), equidistant from each other, and allowed to explore the objects for 5 min. Thirty minutes later, one copy of the familiar object (A3) and a new object (B) were placed in the same location as during the training trial. Each rat was placed in the apparatus for 5 minutes, and the time spent exploring each object was recorded. The discrimination index was calculated as the difference in time exploring the novel and the familiar objects, expressed as the percentage ratio of the total time spent exploring both objects (Schiavi et al., 2022).

### Statistical Analysis

Data are expressed as mean±SEM and were analyzed by two-way ANOVA, with genotype (WT or Fmr1-^{Δ}exon 8) and treatment (psilocybin or vehicle) as factors. Tukey's post hoc test was used for individual group comparisons. The accepted value for significance was set at p<0.05. Data were analyzed using GraphPad Prism 8 software.

### Results

Experiment 1: Acute Intraperitoneal Treatment with Psilocybin Mitigates the Cognitive Deficits Displayed by Fmr1-^{Δ}exon 8 Rats

The Fmr1-^{Δ}exon 8 rat is a recognized genetic model of FXS (Golden et al., 2019; Schiavi et al., 2022). The key trait of FXS is cognitive dysfunction (Hagerman et al., 2017). Accordingly, animal models of FXS are impaired in a wide range of cognitive tests (Melancia and Trezza, 2018).

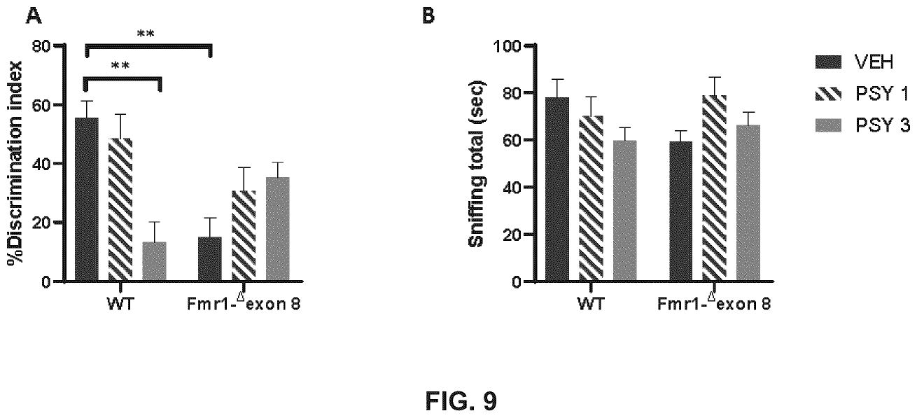

In particular, we have recently reported that juvenile and adult Fmr1-^{Δ}exon 8 rats show cognitive impairment in recognition memory processing, manifested as an inability to discriminate novel from familiar objects (Schiavi et al., 2022). Experiment 1 showed that acute i.p. administration of psilocybin, at the doses of 1 and 3 mg/kg eight days before testing, only mitigated the cognitive deficits displayed by Fmr1-^{Δ}exon 8 rats. In particular, the two-way ANOVA analysis performed on the parameters recorded during the test gave the following results: % Discrimination index (F_{(genotype)1,69}=4.54, p<0.05; F_{(treatment)2,69}=2.67, p=n.s.; F_{(genotype×treatment) 2,69}=11.08, p<0.001) (FIG. 9A); Time sniffing total (F_{(genotype) 1,69}=0.057, p=n.s.; F_{(treatment)2,69}=1.455, p=n.s.; F_{(genotype×treatment) 2,69}=2.41, p=n.s) (FIG. 9B). Post hoc analysis confirmed the presence of a robust cognitive deficit in Fmr1-^{Δ}exon 8 rats as expressed by their lower discrimination index as compared to WT controls (p<0.01, FIG. 9A). Single psilocybin administration mitigated this deficit, as the performance of Fmr1-^{Δ}exon 8 rats treated with psilocybin did not differ statistically from WT controls. Interestingly, psilocybin at the dose of 1 mg/kg did not affect the performance of WT animals; conversely, WT animals treated with psilocybin at the dose of 3 mg/kg showed poorer cognitive performance when compared to WT rats treated with vehicle (p<0.001, FIG. 9A). For all the experimental groups, no alterations were found in the total time spent by the animals exploring the objects during the test phase, thus suggesting an intact object exploratory activity (FIG. 9B).

Experiment 2: Repeated Intraperitoneal Treatment with Psilocybin Normalizes the Cognitive Deficits Displayed by Fmr1-^{Δ}exon 8 Rats

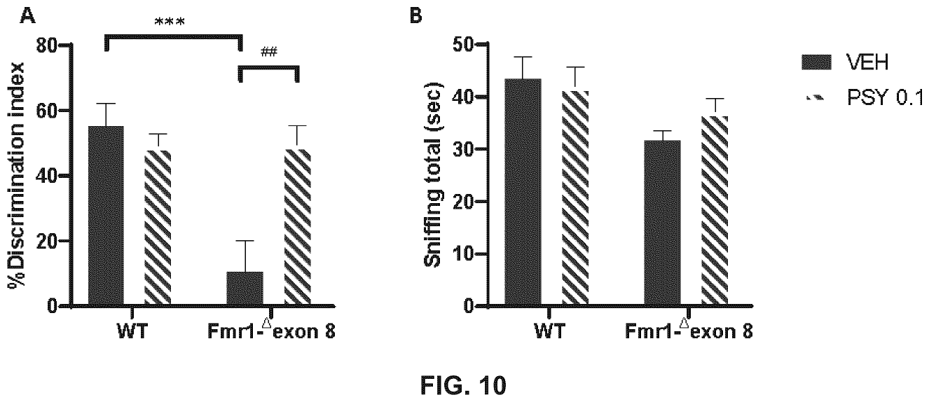

Experiment 2 tested whether repeated psilocybin microdosing (0.1 mg/kg i.p. on alternate days) would rescue the cognitive deficits displayed by Fmr1-^{Δ}exon 8 rats, without affecting the performance of WT animals. The two-way ANOVA analysis performed on the parameters recorded during the test gave the following results: % Discrimination index (F_{(genotype) 1,32}=10.23, p=0.0031; F_{(treatment) 1,32}=5.75, p=0.0225; F_{(genotype×treatment) 1,32}=10.53; p=0.0028) (FIG. 10A); Time sniffing total (F_{(genotype) 1,32}=6.18, p=0.0183; F_{(treatment) 1,32}=0.36, p=0.5528; F_{(genotype×treatment) 1,32}=1.03, p=0.3176) (FIG. 10B). Posthoc analysis revealed that the cognitive impairment displayed by Fmr1-^{Δ}exon 8 rats (p<0.001 vs WT animals treated with vehicle) was rescued in Fmr1-^{Δ}exon 8 rats repeatedly treated with psilocybin (p<0.01 vs Fmr1-^{Δ}exon 8 treated with vehicle, FIG. 10A). As for the previous experiments, no significant differences were found in the total time spent by the animals exploring the objects during the test phase (FIG. 10B), indicating an intact object exploratory activity in all the experimental groups.

Experiment 3: Repeated Oral Treatment with Psilocybin Reversed the Cognitive Deficits Displayed by Fmr1-^{Δ}exon 8 Rats

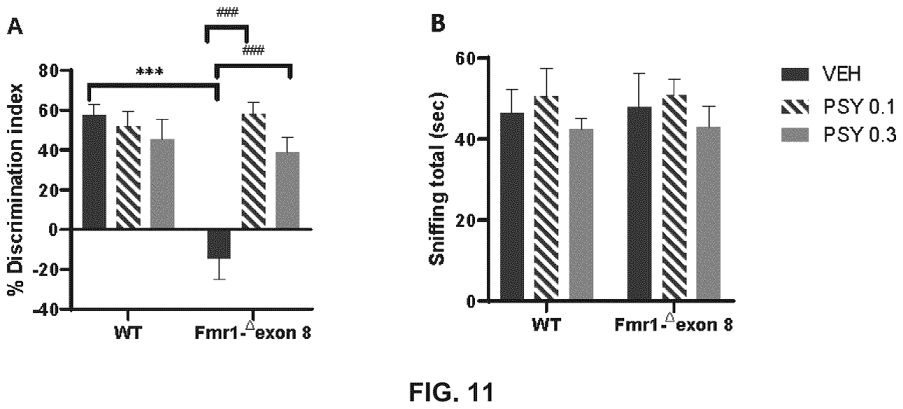

Experiment 3 confirmed the efficacy of psilocybin microdoses in recovering the cognitive deficit displayed by Fmr1-^{Δ}exon 8 animals while performing a novel object recognition task. The two-way ANOVA analysis showed the following results: % Discrimination index (F(genotype) 1,49-13.56, p<0.001; F_{(treatment)2,49}=8.59, p<0.001; F_{(genotype×treatment)2,49}=13.07, p<0.001) (FIG. 11A); Time sniffing total (F_{(genotype) 1,49}=0.030, p=n.s.; F_{(treatment) 2,49}=1.064, p=n.s.; F_{(genotype×treatment) 2,49}=0.004, p=n.s.) (FIG. 11B). Post hoc analysis revealed that the marked cognitive impairment displayed by Fmr1-^{Δ}exon 8 rats (p<0.001 vs WT animals treated with vehicle) was reverted by repeated oral administration of psilocybin at both 0.1 and 0.3 mg/kg doses (p<0.001 vs Fmr1-^{Δ}exon 8 treated with vehicle, FIG. 11A). Again, no significant differences were detected in the total time spent by animals exploring the objects during the test phase (FIG. 11B), confirming a proper exploratory activity toward the objects in all the experimental groups.

### DISCUSSION

This study provides evidence of the beneficial effects of different schedules of psilocybin treatment in mitigating the cognitive deficit observed in a rat model of FXS. Specifically, our results revealed that systemic and oral administration of psilocybin microdoses normalizes the aberrant cognitive performances displayed by adolescent Fmr1-^{Δ}exon 8 rats in the novel object recognition test, thus supporting the hypothesis that serotonin modulating drugs such as psilocybin may be useful to ameliorate ASD-related cognitive deficits.

In recent years, the therapeutic potential of psychedelic drugs is gaining scientific interest, especially regarding their potential application in the treatment of mental diseases (Belouin and Henningfield, 2018; De Gregorio et al., 2021; Rucker et al., 2018). Psychedelics, including psilocybin, exert their pharmacological effects primarily through the serotonergic system, acting mainly as agonists of the 5-HT_{2A} receptor (Vollenweider et al., 2007; Vollenweider and Kometer, 2010). The serotonin system is a logical candidate for involvement in ASD and co-occurring conditions including FXS, due to its pleiotropic role across multiple brain systems along development (Muller et al., 2016; Wirth et al., 2017). Reflecting its dominating neurodevelopmental control, serotonin levels peak in neurotypical children between ages 2 and 5 years, when brain serotonin synthesis capacity reaches twice the levels found in the adult brain (Chugani et al., 1998). In contrast, serotonin levels are much lower in children with developmental delay (Chugani, 2002) and increase gradually, missing the peak of early childhood. This has suggested that therapeutic intervention with selective serotonin reuptake inhibitors (SSRIs) may be more beneficial in treating ASD-symptoms (i.e., hyperactivity, anxiety, aggression) during early childhood as opposed to later in life (Hagerman et al., 2009; Hanson and Hagerman, 2014).

However, SSRIs have side effects that may worsen symptoms. Psilocybin on the other hand has an excellent safety profile (Hendricks et al., 2015; Rucker et al., 2018) and may prove to be superior to existing methods to therapeutically modulate the serotonergic system. For instance, clinical evidence showed that psilocybin exerts positive effects on some aspects of social cognition that are important in relation to the deficits observed in ASD (Pokorny et al., 2017; Preller et al., 2016) and produces marked pro-social effects (Forstmann et al., 2020; Smigielski et al., 2019). In line with this evidence, Hibicke and colleagues recently demonstrated the antidepressant effect of psilocybin in Wistar rats (Hibicke et al., 2020), and a study by Mollinedo-Gajate and colleagues still under review has pointed out how psilocybin rescues the social alterations displayed by mice prenatally exposed to valproic acid (Mollinedo-Gajate et al., https://doi.org/10.1101/2020.09.09.289348), a widely used environmental model of ASD (Servadio et al., 2018; Tartaglione et al., 2019). Although significant research has been carried out, there have been few studies specifically in the FXS population and the precise role of serotonergic signaling in FXS is still under active investigation.

To address this issue, we tested the effects of psilocybin treatment in the cognitive dysfunctions displayed by adolescent Fmr1-^{Δ}exon 8 animals, by testing different schedules of psilocybin administration. Cognitive dysfunction is a key trait in FXS (Hagerman et al., 2017) and it is a common co-morbid feature of non-syndromic ASD (Lai et al., 2014). Accordingly, several preclinical studies have shown that Fmr1-KO mice display cognitive impairments (Ding et al., 2014; King and Jope, 2013; Melancia and Trezza, 2018; Ventura et al., 2004) and we recently generalized these findings to Fmr1-^{Δ}exon 8 rats (Schiavi et al., 2022). Specifically, we first tested whether single systemic administrations of psilocybin at the doses of 1 and 3 mg/Kg would correct aberrant object discrimination displayed by Fmr1-^{Δ}exon 8 rats. Interestingly, we found that single systemic administration of psilocybin at the dose of 1 mg/kg mitigated (but not completely rescued) the cognitive deficits displayed by Fmr1-^{Δ}exon 8 animals while performing the novel object recognition task. The drug was administered eight days before the animals performed the behavioral task to prevent any potential psychedelic effect at the time of testing. Conversely, we found a detrimental effect of the higher dose of psilocybin tested (3 mg/kg) in WT animals, since their cognitive performance in the novel object recognition task got worse: thus, the values of their discrimination index, which assesses the preference that rodents display for investigating novel rather than familiar objects (Manduca et al., 2017), appeared to be similar to the values of Fmr1-^{Δ}exon 8 rats. This prompted us to investigate the effects of lower doses of psilocybin along with different schedules of administration.

Clinical studies highlighted a more effective therapeutic potential of psychedelic substances administered through microdoses (Hasler et al., 2004; Kaertner et al., 2021; Kuypers, 2020). Microdoses appear to be more beneficial compared to standard doses as they do not seem to give perceptive alterations (Ona and Bouso, 2020; Vollenweider and Kometer, 2010). However, the exact parameters determining a correct posology of a microdose have yet to be firmly articulated. For instance, microdosing has been generally described to involve successive administration within a limited time window of doses of psychedelics that do not impair normal functioning and are predominantly sub-sensorium (Kuypers et al., 2019). Based on anecdotal evidence of human microdosing following a popular protocol of repeated dosing with interspersing rest days (Fadim, 2011; Lea et al., 2020), we treated Fmr1-^{Δ}exon 8 rats with repeated systemic administration of psilocybin every other day within five days at the dose of 0.1 mg/kg (i.p.) with the last treatment given five days before testing to prevent any hallucinogenic effect at the time of behavioral performance. Our results showed that this schedule of psilocybin administration was able to fully rescue the cognitive impairment displayed by Fmr1-^{Δ}exon 8 rats while performing a novel object recognition task, without affecting the performance of WT animals. To our knowledge, this is the first evidence showing psychedelic microdosing benefits in the cognitive deficits displayed by a rat model of FXS.

To increase the translational value of our findings, we finally decided to assess the effects of repeated oral administration of 0.1 and 0.3 mg/kg psilocybin microdoses, for two weeks every other day, in our Fmr1-^{Δ}exon 8 rats. The animals were tested five days after the last treatment, to prevent any hallucinogenic effect of the drug at the time of behavioral performance. As for the previous experiment, we found that psilocybin restored the cognitive impairments displayed by Fmr1-^{Δ}exon 8 rats at either the two doses tested (0.1 mg/kg and 0.3 mg/kg), without affecting the performance of WT animals. Importantly, psilocybin treatment did not induce any sign of discomfort/intoxication in the animals, neither immediately nor days after administration, suggesting a good safety profile of the proposed schedule of drug administration. This is in line with evidence form Higgins and colleagues showing that psilocybin administration at the dose of 0.1 mg/kg is devoid of side effects on motor activity or stereotypies in treated rats (Higgins et al., 2021). In our opinion, this is important to remark since these results on efficacy and safety of this schedule of psilocybin administration could guide future studies examining the possibility of longer dosing schedules and they anticipated that no adverse events are associated with the microdosing regimen proposed herein.

Since both psilocybin and its active metabolite psilocin act as agonists at serotonin receptors, with various binding affinities across subtypes, it is tempting to speculate that the benefits we here obtained from psilocybin treatment may be due to its binding to the 5HT₂ₐreceptors (Gill et al., 2020; Lowe et al., 2021; Passie et al., 2002). For instance, 5HT₂ₐ receptors are found in various brain areas such as the prefrontal cortex and thalamus which are involved in the mechanisms related to the onset of psychotic symptoms (Lowe et al., 2021). Some studies suggest that the activation of the 5HT₂ₐ receptors causes neuroplastic changes that lead to the down-regulation of the receptor itself, leading to anxiolytic and antidepressant effects (Lowe et al., 2021). In particular, several studies assessed its antidepressant potential in patients with major depressive disorder and its anxiolytic properties in patients with cancer-related anxiety and depression (Davis et al., 2021; Ross et al., 2016). Starting from the assumption that symptoms such as anxiety and depression are comorbid features of ASD (Hagerman and Hagerman, 2016; Hollocks et al., 2019; White et al., 2018; Zafarullah and Tassone, 2019) and that clinical and preclinical studies show an alteration in the production and regulation of serotonin in ASD subjects (Chugani, 2004; Launay et al., 1988; Takumi et al., 2020) one can argue that psilocybin treatment could restore the unbalanced serotonin pathways in our animal genetic model of ASD, leading to a beneficial effects on cognitive disturbances. However, this remains an interesting observation that requires further investigation, at least in our experimental settings.

### CONCLUSIONS

With the increase in the rate of neurodevelopmental disorders, more studies are warranted to assess psilocybin safety to aid clinicians and policymakers in evidence-informed decision-making. Our results contribute to highlight the therapeutic potential of psilocybin in treating cognitive dysfunction in a genetic animal model of ASD that is also a model of FXS. However, this study offers many challenges to overcome: 1) different time and dose windows of psilocybin exposure should be thoroughly investigated. For instance, infancy represents an important period for postnatal development of the offspring and the effects of psilocybin at this critical time window seek further investigation; 2) the use of other environmental/genetic animal models of ASD should be encouraged to increase the validity of our results; 3) since the mechanisms underlying the beneficial effects of psilocybin remain elusive, testing serotonin antagonists should be increased.

### Example 4. Effects of Psilocybin in the Valproic Acid Rat Model of Autism Spectrum Disorder Background

Prenatal exposure to the antiepileptic drug valproic acid (VPA) induces autistic-like core behavioral symptoms in both humans and rodents, which makes it a good model to study the neural underpinnings of autism spectrum disorder (ASD). Rats prenatally exposed to VPA show profound deficits in the social domain, together with stereotypies and anxiety-like behaviors (Servadio et al. 2016).

### Aim

We tested whether acute administration of psilocybin (1 mg/kg, intraperitoneal (i.p.) was able to mitigate the anxiety-like behavior displayed by VPA-exposed rats in the elevated plus maze test, a prototypical animal model of anxiety.

### Methods

a. Animals.

Female Wistar rats (Charles River), weighing 250±15 g, were mated overnight. The morning when spermatozoa were found was designated as gestational day 1 (GD1). Pregnant rats were singly housed in Macrolon cages (401×26w x 20h cm), under controlled conditions (temperature 20-21° C., 55-65% relative humidity and 12/12h light cycle with lights on at 07:00 a.m.). Food and water were available ad libitum. On gestational day 12.5, females received a single intraperitoneal injection of either sodium valproate (VPA) or saline (SAL). VPA (Cayman) was dissolved in saline at a concentration of 250 mg/ml and administered at a dose (500 mg/kg) and time (GD 12.5) that have been shown to induce autistic-like behavioral changes in the offspring (Servadio et al., 2016). Newborn litters found up to 5.00 p.m. were considered to be born on that day (Postnatal day (PND) 0). On PND 1, the litters were culled to eight animals (six males and two females). On PND 21, the pups were weaned and housed in groups of three. Experiments were carried out on the male offspring at adolescence (PND 35).

The experiments were approved by the Animal Welfare Committee of Roma Tre University, by the Italian Ministry of Health (authorization number: 988/2020-PR) (Rome, Italy) and performed in agreement with the ARRIVE (Animals in Research: Reporting In Vivo Experiments) guidelines, the guidelines of the Italian Ministry of Health (D. L. 26/14) and the European Community Directive 2010/63/EU.

b. Elevated Plus-Maze Test

The apparatus comprised two open and two closed arms (501×10×40 h cm) that extended from a common central platform (101×10 w cm). Rats were individually placed on the central platform of the maze for 5 min. Each session was recorded with a camera positioned above the apparatus for subsequent behavioral analysis performed using the Observer 3.0 software (Noldus Information Technology). The following parameters were analyzed:

% time spent in the open arms (% TO): (seconds spent on the open arms of the maze/seconds spent on the open+closed arms)×100; 1.

% open arm entries (% OE): (the number of entries into the open arms of the maze/number of entries into open+closed arms)×100. 2.

d. Statistical Analysis

Data are expressed as mean±SEM. To assess the effects of prenatal VPA exposure and psilocybin on behavioral parameters, data were analyzed by two-way ANOVA, with prenatal treatment and postnatal treatment as factors. The Tukey's post hoc test was used for individual group comparisons.

e. Treatment

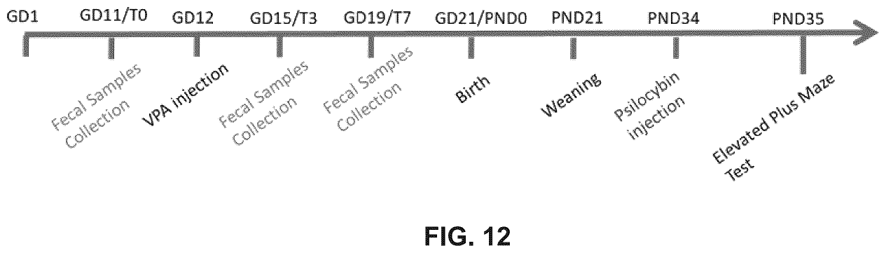

Psilocybin was dissolved in saline and given i.p. to either VPA- or Saline-exposed offspring at 1 mg/kg on PND 34. The following day, the animals were tested in the elevated plus-maze test. The timeline of the experiment is shown in FIG. 12.

### Results

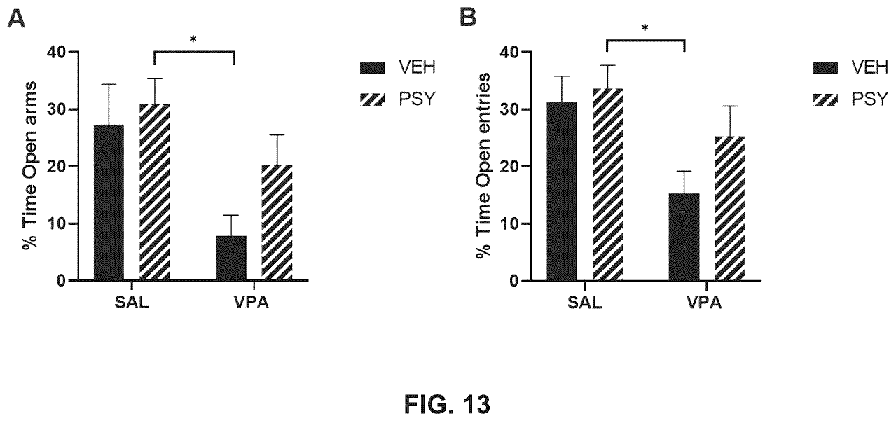

Results are shown in FIG. 13. As expected, VPA-exposed rats showed an anxiety-like phenotype, as they spent less time in the open arms of the maze (Panel A) and made less entries in the open arms (Panel B). Psilocybin treatment reversed the anxiety-like behavior displayed by VPA-exposed rats.

### REFERENCES

Ashwood P, Enstrom A, Krakowiak P, Hertz-Picciotto I, Hansen R L, Croen L A, Ozonoff S, Pessah IN, Van de Water J. Decreased transforming growth factor betal in autism: a potential link between immune dysregulation and impairment in clinical behavioral outcomes. J Neuroimmunol. 2008 Nov. 15; 204(1-2):149-53.
Ashwood P, Krakowiak P, Hertz-Picciotto I, Hansen R, Pessah I, Van de Water J. Elevated plasma cytokines in autism spectrum disorders provide evidence of immune dysfunction and are associated with impaired behavioral outcome. Brain Behav Immun. 2011 January;25(1):40-5.
Bardoni, B., Davidovic, L., Bensaid, M., Khandjian, E. W., 2006. The fragile X syndrome: exploring its molecular basis and seeking a treatment. Expert Rev Mol Med 8, 1-16.
Belouin, S. J., Henningfield, J. E., 2018. Psychedelics: Where we are now, why we got here, what we must do. Neuropharmacology 142, 7-19.
Blaszkiewicz M, Willows J W, Johnson C P, Townsend K L. The Importance of Peripheral Nerves in Adipose Tissue for the Regulation of Energy Balance. Biology (Basel). 2019 Feb. 12; 8(1):10.
Bondoux, D., Luong, C., Dreux, C., 1988. Serotonin metabolism and other biochemical parameters in infantile autism. A controlled study of 22 autistic children. Neuropsychobiology 20, 1-11.
Breit, S. et al. 2018. Vagus nerve as modulator of the brain-gut axis in psychiatric and inflammatory disorders. Fron. in Psych. Review. 13 Mar. 2018.
Breit, S. et al. 2018. Vagus nerve as modulator of the brain-gut axis in psychiatric and inflammatory disorders. Fron. in Psych. Review. 13 Mar. 2018.
Caraci F, Spampinato S F, Morgese M G, Tascedda F, Salluzzo M G, Giambirtone M C, Caruso G, Munafo A, Torrisi S A, Leggio G M, Trabace L, Nicoletti F, Drago F, Sortino M A, Copani A. Neurobiological links between depression and AD: The role of TGF-β1 signaling as a new pharmacological target. Pharmacol Res. 2018 April;130:374-384
Chugani, D. C., 2002. Role of altered brain serotonin mechanisms in autism. Mol Psychiatry 7 Suppl 2, 516-17.
Chugani, D. C., 2004. Serotonin in autism and pediatric epilepsies. Ment Retard Dev Disabil Res Rev 10, 112-116.
Chugani, D. C., Muzik, O., Chakraborty, P., Mangner, T., Chugani, H. T., 1998. Human brain serotonin synthesis capacity measured in vivo with alpha-[C-11]methyl-L-tryptophan. Synapse 28, 33-43.
Costa, L., Sardone, L. M., Lacivita, E., Leopoldo, M., Ciranna, L., 2015. Novel agonists for serotonin 5-HT7 receptors reverse metabotropic glutamate receptor-mediated long-term depression in the hippocampus of wild-type and Fmr1 KO mice, a model of Fragile X Syndrome. Front Behav Neurosci 9, 65.
Daly D M, Park S J, Valinsky WC, Beyak MJ. Impaired intestinal afferent nerve satiety signaling and vagal afferent excitability in diet induced obesity in the mouse. Jphysiol 2011; 589:2857-70.
Darnell, J. C., Klann, E., 2013. The translation of translational control by FMRP: therapeutic targets for FXS. Nat Neurosci 16, 1530-1536.
Davis, A. K., Barrett, F. S., May, D. G., Cosimano, M. P., Sepeda, N. D., Johnson, M. W., Finan, P. H., Griffiths, R. R., 2021. Effects of Psilocybin-Assisted Therapy on Major Depressive Disorder: A Randomized Clinical Trial. JAMA Psychiatry 78, 481-489.
De Gregorio, D., Aguilar-Valles, A., Preller, K. H., Heifets, B. D., Hibicke, M., Mitchell, J., Gobbi, G., 2021. Hallucinogens in Mental Health: Preclinical and Clinical Studies on LSD, Psilocybin, MDMA, and Ketamine. J Neurosci 41, 891-900.
deLartigue G, Barbier de la Serre C, Espero E, Lee J, Raybould H E. Diet-induced obesity leads to the development of leptin resistance in vagal afferent neurons. AmJPhysiol Endocrinology and Metabolism 2011;30(1):E187-95.
de Lartigue G. Role of the vagus nerve in the development and treatment of diet-induced obesity. J Physiol. 2016; 594(20):5791-5815. doi:10.1113/JP271538
Di Marco, B., Bonaccorso, C. M., Aloisi, E., D'Antoni, S. & Catania, M. V. Neuro-Inflammatory Mechanisms in Developmental Disorders Associated with Intellectual Disability and Autism Spectrum Disorder: A Neuro-Immune Perspective. CNS Neurol Disord Drug Targets 15, 448-463 (2016).
Ding, Q., Sethna, F., Wang, H., 2014. Behavioral analysis of male and female Fmr1 knockout mice on C57BL/6 background. Behav Brain Res 271, 72-78.
El-Ansary A, Bjorklund G, Chirumbolo S, Alnakhli O M. Predictive value of selected biomarkers related to metabolism and oxidative stress in children with autism spectrum disorder. Metab Brain Dis. 2017 August; 32(4):1209-1221.
Ellulu M S, Patimah I, Khaza′ai H, Rahmat A, Abed Y. Obesity and inflammation: the linking mechanism and the complications. Arch Med Sci. 2017; 13(4):851-863.
Evrensel A, Ceylan M E. The gut-brain axis: the missing link in depression. Clin Psychopharmacol Neurosci (2015) 13:239-44.
Fadim, J., 2011. The Psychedelic Explorer's Guide: Safe, Therapeutic, and Sacred Journeys.
Forstmann, M., Yudkin, D. A., Prosser, A. M. B., Heller, S. M., Crockett, M. J., 2020. Transformative experience and social connectedness mediate the mood-enhancing effects of psychedelic use in naturalistic settings. Proc Natl Acad Sci USA 117, 2338-2346.
Fujisaka, S. et al. Regulatory mechanisms for adipose tissue M1 and M2 macrophages in diet-induced obese mice. Diabetes 58, 2574-2582 (2009)
Garcia-Romeu, A. et al. 2021. J. Psychopharmacol. 35:353-361.
Gill, H., Gill, B., Chen-Li, D., El-Halabi, S., Rodrigues, N. B., Cha, D. S., Lipsitz, O., Lee, Y., Rosenblat, J. D., Majeed, A., Mansur, R. B., Nasri, F., Ho, R., McIntyre, R. S., 2020. The emerging role of psilocybin and MDMA in the treatment of mental illness. Expert Rev Neurother 20, 1263-1273.
Golden, C. E. M., Breen, M. S., Koro, L., Sonar, S., Niblo, K., Browne, A., Burlant, N., Di Marino, D., De Rubeis, S., Baxter, M. G., Buxbaum, J. D., Harony-Nicolas, H., 2019. Deletion of the KH1 Domain of Fmr1 Leads to Transcriptional Alterations and Attentional Deficits in Rats. Cereb Cortex 29, 2228-2244.
Griffiths, R. R. et al. 2011. Psychopharmacology 218:649-665.
Hagemann, D. et al. 2003. Appetite regulation by ghrelin— a novel neuro-endocrine gastric peptide hormone in the gut-brain-axis. Z Gastroenterol. 41:929-36.
Hagerman, R. J., Berry-Kravis, E., Hazlett, H. C., Bailey, D. B., Jr., Moine, H., Kooy, R. F., Tassone, F., Gantois, I., Sonenberg, N., Mandel, J. L., Hagerman, P. J., 2017. Fragile X syndrome. Nat Rev Dis Primers 3, 17065.
Hagerman, R. J., Berry-Kravis, E., Kaufmann, W. E., Ono, M. Y., Tartaglia, N., Lachiewicz, A., Kronk, R., Delahunty, C., Hessl, D., Visootsak, J., Picker, J., Gane, L., Tranfaglia, M., 2009. Advances in the treatment of fragile X syndrome. Pediatrics 123, 378-390.
Hagerman, R. J., Hagerman, P., 2016. Fragile X-associated tremor/ataxia syndrome-features, mechanisms and management. Nat Rev Neurol 12, 403-412.
Han Y M, Cheung W K, Wong C K, Sze S L, Cheng T W, Yeung M K, Chan A S. Distinct Cytokine and Chemokine Profiles in Autism Spectrum Disorders. Front Immunol. 2017 Jan. 23; 8:11.
Hanson, A. C., Hagerman, R. J., 2014. Serotonin dysregulation in Fragile X Syndrome: implications for treatment. Intractable Rare Dis Res 3, 110-117.
Harris, S. W., Hessl, D., Goodlin-Jones, B., Ferranti, J., Bacalman, S., Barbato, I., Tassone, F., Hagerman, P. J., Herman, H., Hagerman, R. J., 2008. Autism profiles of males with fragile X syndrome. Am J Ment Retard 113, 427-438.
Haruwaka K, Ikegami A, Tachibana Y, Ohno N, Konishi H, Hashimoto A, Matsumoto M, Kato D, Ono R, Kiyama H, Moorhouse AJ, Nabekura J, Wake H. Dual microglia effects on blood brain barrier permeability induced by systemic inflammation. Nat Commun. 2019 Dec. 20; 10(1):5816.
Hasler, F., Grimberg, U., Benz, M. A., Huber, T., Vollenweider, F. X., 2004. Acute psychological and physiological effects of psilocybin in healthy humans: a double-blind, placebo-controlled dose-effect study. Psychopharmacology (Berl) 172, 145-156.
Henchcliffe C, Beal M E. Mitochondrial biology and oxidative stress in Parkinson disease pathogenesis. Nat Clin Pract Neurol. 2008;4 (11):600-609.
Hendricks, P. S., Thorne, C. B., Clark, C. B., Coombs, D. W., Johnson, M. W., 2015. Classic psychedelic use is associated with reduced psychological distress and suicidality in the United States adult population. J Psychopharmacol 29, 280-288.
Heisler L K, Cowley M A, Tecott L H, Fan W, Low M J, Smart J L, Rubinstein M, Tatro J B, Marcus I N, Holstege H, Lee C E, Cone R D, Elmquist J K. Activation of central melanocortin pathways by fenfluramine. Science. 2002 Jul. 26; 297(5581):609-11.
Hernandez, R. N., Feinberg, R. L., Vaurio, R., Passanante, N. M., Thompson, R. E., Kaufmann, W. E., 2009. Autism spectrum disorder in fragile X syndrome: a longitudinal evaluation. Am J Med Genet A 149A, 1125-1137.
Heuer L S, Croen L A, Jones K L, Yoshida C K, Hansen R L, Yolken R, Zerbo 0, DeLorenze G, Kharrazi M, Ashwood P, Van de Water J. An Exploratory Examination of Neonatal Cytokines and Chemokines as Predictors of Autism Risk: The Early Markers for Autism Study. Biol Psychiatry. 2019 Aug. 15; 86(4):255-264 Huber, K H. et al. 2016. An emerging role for eotaxins in neurodegenerative disease. Clin. Immun. 189: 29-33.
Holscher C. Brain insulin resistance: role in neurodegenerative disease and potential for targeting. Expert Opin Investig Drugs. 2020 April; 29(4):333-348. Jin, C. & Flavell, R. A. Innate sensors of pathogen and stress: linking inflammation to obesity. J. Allergy Clin. Immunol. 132, 287-294 (2013).
Hibicke, M., Landry, A. N., Kramer, H. M., Talman, Z. K., Nichols, C. D., 2020. Psychedelics, but Not Ketamine, Produce Persistent Antidepressant-like Effects in a Rodent Experimental System for the Study of Depression. ACS Chem Neurosci 11, 864-871.
Higgins, G. A., Carroll, N. K., Brown, M., MacMillan, C., Silenieks, L. B., Thevarkunnel, S., Izhakova, J., Magomedova, L., DeLannoy, I., Sellers, E. M., 2021. Low Doses of Psilocybin and Ketamine Enhance Motivation and Attention in Poor Performing Rats: Evidence for an Antidepressant Property. Front Pharmacol 12, 640241.
Hollocks, M. J., Lerh, J. W., Magiati, I., Meiser-Stedman, R., Brugha, T. S., 2019. Anxiety and depression in adults with autism spectrum disorder: a systematic review and meta-analysis. Psychol Med 49, 559-572.
Hoyer S, Oesterreich K, Wagner O. Glucose metabolism as the site of the primary abnormality in early-onset dementia of Alzheimer type? J Neurol. 1988; 235(3):143-148.
Kempuraj D, Thangavel R, Natteru P A, et al. Neuroinflammation Induces Neurodegeneration. J Neurol Neurosurg Spine. 2016;1(1):1003.
Johnson, Michaela & Stecher, Benjamin & Labrie, Viviane & Brundin, Lena & Brundin, Patrik & Org, Patrik & Brundin, P. (2018). Triggers, Facilitators, and Aggravators: Redefining Parkinson's Disease Pathogenesis A New Conceptual Model for Understanding Parkinson's Disease. Trends in Neurosciences.
Kentish S, Li H, Philp LK, O'Donnell T A, Isaacs N J, Young R L, et al. Diet-induced adaptation of vagal afferent function. JPhysiol 2012; 590:209-21. Lobos-Silva, D. et al. 2016. Balancing the immune response in the brain: IL-10 and its Malaca et al. 2020. Int. J. Mol. Sci. 21:9279 (ijms21239279). regulation. J Neuroinflammation. 13(1):297
Kaertner, L. S., Steinborn, M. B., Kettner, H., Spriggs, M. J., Roseman, L., Buchborn, T., Balaet, M., Timmermann, C., Erritzoe, D., Carhart-Harris, R. L., 2021. Positive expectations predict improved mental-health outcomes linked to psychedelic microdosing. Sci Rep 11, 1941.
Khemakhem A M, Frye R E, El-Ansary A, Al-Ayadhi L, Bacha AB. Novel biomarkers of metabolic dysfunction is autism spectrum disorder: potential for biological diagnostic markers. Metab Brain Dis. 2017 December; 32(6):1983-1997.
Kilkenny, C., Browne, W. J., Cuthill, I. C., Emerson, M., Altman, D. G., 2010. Improving bioscience research reporting: the ARRIVE guidelines for reporting animal research. PLoS Biol 8, e1000412.
King, M. K., Jope, R. S., 2013. Lithium treatment alleviates impaired cognition in a mouse model of fragile X syndrome. Genes Brain Behav 12, 723-731.
Kuypers, K. P., Ng, L., Erritzoe, D., Knudsen, G. M., Nichols, C. D., Nichols, D. E., Pani, L., Soula, A., Nutt, D., 2019. Microdosing psychedelics: More questions than answers? An overview and suggestions for future research. J Psychopharmacol 33, 1039-1057.
Kuypers, K. P. C., 2020. The therapeutic potential of microdosing psychedelics in depression. Ther Adv Psychopharmacol 10, 2045125320950567.
Kuypers, K. P. C. 2020. Ther. Adv. Psychopharacol. 10:1-15.
Lai, M. C., Lombardo, M. V., Baron-Cohen, S., 2014. Autism. Lancet 383, 896-910. Launay, J. M., Ferrari, P., Haimart, M., Bursztejn, C., Tabuteau, F., Braconnier, A., Pasques
Lea, T., Amada, N., Jungaberle, H., Schecke, H., Klein, M., 2020. Microdosing psychedelics: Motivations, subjective effects and harm reduction. Int J Drug Policy 75, 102600.
Lesch, K. P., Waider, J., 2012. Serotonin in the modulation of neural plasticity and networks: implications for neurodevelopmental disorders. Neuron 76, 175-191.
Lowe, H., Toyang, N., Steele, B., Valentine, H., Grant, J., Ali, A., Ngwa, W., Gordon, L., 2021. The Therapeutic Potential of Psilocybin. Molecules 26.
Lu J, Zhao J, Meng H, Zhang X. Adipose Tissue-Resident Immune Cells in Obesity and Type 2 Diabetes. Front Immunol. 2019 May 22; 10:1173.
Masi A, Quintana D S, Glozier N, Lloyd A R, Hickie I B, Guastella A J. Cytokine aberrations in autism spectrum disorder: a systematic review and meta-analysis. Mol Psychiatry. 2015 Apr;20(4):440-6.
Manduca, A., Bara, A., Larrieu, T., Lassalle, O., Joffre, C., Laye, S., Manzoni, O. J., 2017. Amplification of mGlu5-Endocannabinoid Signaling Rescues Behavioral and Synaptic Deficits in a Mouse Model of Adolescent and Adult Dietary Polyunsaturated Fatty Acid Imbalance. J Neurosci 37, 6851-6868.
Markopoulos, A., Inserra, A., De Gregorio, D., Gobbi, G., 2021. Evaluating the Potential Use of Serotonergic Psychedelics in Autism Spectrum Disorder. Front Pharmacol 12, 749068.
Masi A, Glozier N, Dale R, Guastella A J. The Immune System, Cytokines, and Biomarkers in Autism Spectrum Disorder. Neurosci Bull. 2017 April; 33(2):194-204. Nicholas, C. R. et al. 2020. J. J. Psychopharmacol. 32:770-778.
Melancia, F., Trezza, V., 2018. Modelling fragile X syndrome in the laboratory setting: A behavioral perspective. Behav Brain Res 350, 149-163.
Muller, C. L., Anacker, A. M. J., Veenstra-VanderWeele, J., 2016. The serotonin system in autism spectrum disorder: From biomarker to animal models. Neuroscience 321, 24-41.
Nutt, D., Erritzoe, D., Carhart-Harris, R., 2020. Psychedelic Psychiatry's Brave New World. Cell 181, 24-28.
Oh C M, Park S, Kim H. Serotonin as a New Therapeutic Target for Diabetes Mellitus and Obesity. Diabetes Metab J. 2016; 40(2):89-98. doi:10.4093/dmj.2016.40.2.89
Ona, G., Bouso, J. C., 2020. Potential safety, benefits, and influence of the placebo effect in microdosing psychedelic drugs: A systematic review. Neurosci Biobehav Rev 119, 194-203.
Owen J A, Punt J, Stranford S A, Jones PP, Kuby J. KubyImmunology. New York: W.H. Freeman, 2013.
Passie, T., Seifert, J., Schneider, U., Emrich, H. M., 2002. The pharmacology of psilocybin. Addict Biol 7, 357-364.
Pavlov V A, Tracey K J. Neural regulation of immunity: molecular mechanisms and clinical translation. Nat Neurosci. 2017 February;20(2):156-166.
Perry V H, Holmes C. Microglial priming in neurodegenerative disease. Nat Rev Neurol. 2014 Apr;10(4):217-24.
Pokorny, T., Preller, K. H., Kometer, M., Dziobek, I., Vollenweider, F. X., 2017. Effect of Psilocybin on Empathy and Moral Decision-Making. Int J Neuropsychopharmacol 20, 747-757.
Preller, K. H., Pokorny, T., Hock, A., Kraehenmann, R., Stampfli, P., Seifritz, E., Scheidegger, M., Vollenweider, F. X., 2016. Effects of serotonin 2A/1A receptor stimulation on social exclusion processing. Proc Natl Acad Sci USA 113, 5119-5124.
Reilly, S. M., & Saltiel, A. R. (2017). Adapting to obesity with adipose tissue inflammation. Nature Reviews Endocrinology, 13(11), 633-643.
Ross, S., Bossis, A., Guss, J., Agin-Liebes, G., Malone, T., Cohen, B., Mennenga, S. E., Belser, A., Kalliontzi, K., Babb, J., Su, Z., Corby, P., Schmidt, B. L., 2016. Rapid and sustained symptom reduction following psilocybin treatment for anxiety and depression in patients with life-threatening cancer: a randomized controlled trial. J Psychopharmacol 30, 1165-1180.
Rucker, J. J. H., Iliff, J., Nutt, D. J., 2018. Psychiatry & the psychedelic drugs. Past, present & future. Neuropharmacology 142, 200-218.
Saad, M. J., Santos, A. & Prada, P. O. Linking gut microbiota and inflammation to obesity and insulin resistance. Physiology (Bethesda) 31, 283-293 (2016).
Sabrina Paganoni, Merit Cudkowicz, and James D Berry Clin Investig (Lond). 2014; 4(7): 605-618.
Schiavi, S., Carbone, E., Melancia, F., Buzzelli, V., Manduca, A., Campolongo, P., Pallottini, V., Trezza, V., 2022. Perinatal supplementation with omega-3 fatty acids corrects the aberrant social and cognitive traits observed in a genetic model of autism based on FMR1 deletion in rats. Nutr Neurosci 25, 898-911.
Sergi D, Renaud J, Simola N, et al. Diabetes, a contemporary risk for Parkinson's disease: epidemiological and cellular evidences. Front Aging Neurosci. 2019; 11:302-311.
Servadio, M., Manduca, A., Melancia, F., Leboffe, L., Schiavi, S., Campolongo, P., Palmery, M., Ascenzi, P., di Masi, A., Trezza, V., 2018. Impaired repair of DNA damage is associated with autistic-like traits in rats prenatally exposed to valproic acid. Eur Neuropsychopharmacol 28, 85-96.
Smigielski, L., Scheidegger, M., Kometer, M., Vollenweider, F. X., 2019. Psilocybin-assisted mindfulness training modulates self-consciousness and brain default mode network connectivity with lasting effects. Neuroimage 196, 207-215.
Srinivasan, K. et al. 2016. Untangling the brain's neuroinflammatory and neurodegenerative transcriptional responses. Nature Communications. DOI: 10.1038.
Strle, K. etal. 2001. Interleukin-10 in the brain. Crit Rev Immunol. 21(5):427-49.
Takumi, T., Tamada, K., Hatanaka, F., Nakai, N., Bolton, P. F., 2020. Behavioral neuroscience of autism. Neurosci Biobehav Rev 110, 60-76.
Tartaglione, A. M., Schiavi, S., Calamandrei, G., Trezza, V., 2019. Prenatal valproate in rodents as a tool to understand the neural underpinnings of social dysfunctions in autism spectrum disorder. Neuropharmacology 159, 107477.
Tecott L H. Serotonin and the orchestration of energy balance. Cell Metab. 2007 Nov;6(5):352-61.
Teixera, A L. et al. 2018. Revisiting the Role of Eotaxin-1/CCL11 in Psychiatric Disorders. Fron. in Psych. Review. 14 Jun. 2018.
Ventura, R., Pascucci, T., Catania, M. V., Musumeci, S. A., Puglisi-Allegra, S., 2004. Object recognition impairment in Fmr1 knockout mice is reversed by amphetamine: involvement of dopamine in the medial prefrontal cortex. Behav Pharmacol 15, 433-442.
Vollenweider, F. X., Csomor, P. A., Knappe, B., Geyer, M. A., Quednow, B. B., 2007. The effects of the preferential 5-HT2A agonist psilocybin on prepulse inhibition of startle in healthy human volunteers depend on interstimulus interval. Neuropsychopharmacology 32, 1876-1887.
Vollenweider, F. X., Kometer, M., 2010. The neurobiology of psychedelic drugs: implications for the treatment of mood disorders. Nat Rev Neurosci 11, 642-651.
White, S. W., Simmons, G. L., Gotham, K. O., Conner, C. M., Smith, I. C., Beck, K. B., Mazefsky, C. A., 2018. Psychosocial Treatments Targeting Anxiety and Depression in Adolescents and Adults on the Autism Spectrum: Review of the Latest Research and Recommended Future Directions. Curr Psychiatry Rep 20, 82.
Wirth, A., Holst, K., Ponimaskin, E., 2017. How serotonin receptors regulate morphogenic signalling in neurons. Prog Neurobiol 151, 35-56.
Wyler S C, Lord C C, Lee S, Elmquist J K, Liu C. Serotonergic Control of Metabolic Homeostasis. Front Cell Neurosci. 2017 Sep. 20; 11:277.
Yabut J M, Crane J D, Green A E, Keating D J, Khan W I, Steinberg G R. Emerging Roles for Serotonin in Regulating Metabolism: New Implications for an Ancient Molecule. Endocr Rev. 2019; 40(4):1092-1107. doi:10.1210/er.2018-00283
Yu J H, Kim M S. Molecular mechanisms of appetite regulation. Diabetes Metab J. 2012; 36(6):391-398.
Zafarullah, M., Tassone, F., 2019. Fragile X-Associated Tremor/Ataxia Syndrome (FXTAS). Methods Mol Biol 1942, 173-189.
Zimering M B, Pulikeyil A T, Myers C E, Pang K C. Serotonin 2A Receptor Autoantibodies Increase in Adult Traumatic Brain Injury In Association with Neurodegeneration. J Endocrinol Diabetes. 2020; 7(1):1-8.
PCT Patent Publication WO 2019/180309

In view of the above, it will be seen that several objectives of the invention are achieved, and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure.

That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.” The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A method of treating a disease or disorder in a patient, the method comprising administering a psychoactive tryptamine derivative to the patient in a manner sufficient to treat the disease or disorder,
wherein the disease or disorder is a neurodevelopmental disease, a neurodegenerative disease, a neurometabolic disease, or anxiety.

2. The method of claim 1, wherein the psychoactive tryptamine derivative is psilocybin baeocystin, aeruginascin, or psilocin.

3. The method of claim 1, wherein the psychoactive tryptamine derivative is administered in a dose below which psychedelic effects are perceived by the patient.

4. The method of claim 1, wherein the disease or disorder is a neurodevelopmental disease.

5. The method of claim 4, wherein the neurodevelopmental disease is autism spectrum disorder, attention deficit hyperactivity disorder (ADHD) or fragile X syndrome.

6. The method of claim 1, wherein the disease or disorder is a neurodegenerative disease.

7. The method of claim 6, wherein the neurodegenerative disease is Alzheimer's disease, Parkinson's disease, multiple sclerosis, or amyotrophic lateral sclerosis.

8. The method of claim 1, wherein the disease or disorder is a neurometabolic disease.

9. The method of claim 8, wherein the neurometabolic disease obesity, type 1 diabetes or type 2 diabetes.

10. A method of diagnosing or monitoring progression or treatment of a disease or disorder in a patient, wherein the disease or disorder is a neurodevelopmental disease, a neurodegenerative disease, a neurometabolic disease, or anxiety, the method comprising
determining mRNA levels in the patient of one or more genes for an inflammatory cytokine, a cytokine target, neurotransmission, serotonin signaling, a membrane channel, DNA damage repair, a growth factor, A2AP, CAPG, and/or NHBS, and
treating the patient for the disease or disorder if the mRNA levels indicate that the patient has the disease or disorder, or
continuing treating the patient for the disease or disorder if the mRNA levels indicate that the patient is responding to the treatment of the disease or disorder.

11. The method of claim 10, wherein the one or more genes is eotaxin, eotaxin-2, eotaxin-3, IL-5, MCP-1, MIP-1α, GROα, RANTES, MCP-3, IL-1B, IL-6, IL-8, IL-12A, TGF-01, TNF-α, IL-4, IL-1β, IL-10, CCR3, NOD1, NLRA, IFNGR, HAAO, NMDAR1, GRIN2B, GRIN3A, GRIN2A, TLR-4, TLR3, TLR1, MAOA, TPH1, TPH2, AADAT, OCTN1, P2X4, P2X7, VDAC3, SLC22A15, SLC22A3, SLC3A2, ZNF365, TRIM26, ZNF827, GDNF, PDGF-A, NGF, FGF-13, GIF, A2AP, CAPG, HMBS, or any combination thereof.

12. A method of developing a treatment of a disease or disorder in a patient, wherein the disease or disorder is neurodevelopmental disease, a neurodegenerative disease, a neurometabolic disease or anxiety, the method comprising
treating more than one patient having the disease or disorder with the treatment, and
determining mRNA levels in the patient of one or more genes for an inflammatory cytokine, a cytokine target, neurotransmission, serotonin signaling, a membrane channel, DNA damage repair, a growth factor, A2AP, CAPG, and/or NHBS, wherein
if the mRNA levels indicate that the treatment alleviates a symptom of the disease or disorder, development of the treatment is continued, and
if the mRNA levels indicate that the treatment does not alleviate a symptom of the disease or disorder, development of the treatment is discontinued.

13. The method of claim 12, wherein the treatment is administration of a medication.

14. The method of claim 13, wherein the medication is a psychoactive tryptamine derivative.

15. The method of claim 14, wherein the psychoactive tryptamine derivative is psilocybin baeocystin, aeruginascin, or psilocin.

16. The method of claim 14, wherein the psychoactive tryptamine derivative is administered in a dose below which psychoactive effects are perceived by the patient.

17. (canceled)

18. The method of claim 12, wherein the disease or disorder is autism spectrum disorder, attention deficit hyperactivity disorder (ADHD) or fragile X syndrome.

19. (canceled)

20. The method of claim 12, wherein the disease or disorder is Alzheimer's disease, Parkinson's disease, multiple sclerosis, or amyotrophic lateral sclerosis.

21. (canceled)

22. The method of claim 12, wherein the disease or disorder is obesity, type 1 diabetes or type 2 diabetes.
